(19) United States
(12) Patent Application Publication
(10) Pub. No.: US 2016/0374997 A1
(43) Pub. Date: Dec. 29, 2016

(54) ANTIBIOTIC COMPOUNDS THAT INHIBIT BACTERIAL PROTEIN SYNTHESIS

(71) Applicant: The Board of Regents of The University of Texas System, Austin, TX (US)

(72) Inventors: James M. Bullard, Edinburg, TX (US); Yanmei Hu, Edinburg, TX (US); Stephanie O. Palmer, McAllen, TX (US)

(73) Assignee: Board of Regents of the University of Texas System

(21) Appl. No.: 15/196,225

(22) Filed: Jun. 29, 2016

Related U.S. Application Data
(62) Division of application No. 14/611,606, filed on Feb. 2, 2015.

(51) Int. Cl. A61K 31/433; A61K 31/381; A61K 31/426

(52) U.S. Cl. CPC A61K 31/433; A61K 31/426; A61K 31/381

(57) ABSTRACT

An aminoacylation/translation (AIT) system based on the protein synthesis system from the pathogen Pseudomonas aeruginosa, was used to screen chemical compounds for identifying inhibitors of protein synthesis. This system includes elongation factors: EF-Tu, EF-Ts and EF-G, aminoacyl tRNA synthetase (aaRS) specific for phenylalanine, PheRS, and ribosomes isolated from cultures of Pseudomonas aeruginosa. Compounds identified using this assay have been shown to contain broad spectrum activity against both Gram+ and Gram− pathogens. Methods of using the identified compounds, as well as derivatives and analogues of these compounds, as antimicrobial agents against bacterial infections are described.

### PRIORITY CLAIM

The present application is a division of U.S. patent application Ser. No. 14/611,606, filed Feb. 2, 2015, which is incorporated herein by reference.

### STATEMENT REGARDING FEDERALLY SPONSORED RESEARCH OR DEVELOPMENT

This invention was made with government support under Grant No. 1SC3GM098173-01A1 awarded by the National Institutes of Health-National Institutes of General Medical Science. The government has certain rights in the invention.

### BACKGROUND OF THE INVENTION

1. Field of the Invention

The invention generally relates to antibiotic compounds and compositions.

2. Description of the Relevant Art

Bacterial infections continue to represent a major worldwide health problem. Infections range from the relatively innocuous, such as skin rashes and common ear infections in infants, to serious and potentially lethal infections in immune-compromised patients. Resistance to antibacterial agents has increased in many pathogenic bacteria and can occur through a variety of mechanisms, such as target mutation, induction of efflux pumps, or induction of metabolic pathways leading to the degradation of the compound. Resistance developed in one cell can be transferred to other bacteria by horizontal gene transfer. The need for new antibiotics to address the increase in resistance has become critical.

Antibacterial agents generally interfere with cellular processes that are essential for the survival of the cell. For both naturally occurring and synthetic antibiotics, protein synthesis is a major target of antibiotic action. Bacterial protein synthesis inhibitors include the macrolides (e.g., erythromycin, clarithromycin, and azithromycin), clindamycin, chloramphenicol, the aminoglycosides (e.g., streptomycin, gentamicin, and amikacin), and the tetracyclines. The newest class of antibacterials, the synthetic oxazolidinones (exemplified by linezolid, the only novel and approved ribosomal inhibitor), also inhibit protein synthesis. Protein synthesis is the cellular process most frequently targeted by naturally occurring antibiotic compounds providing compelling evolutionary evidence for the susceptibility of this process to antibiotic intervention.

Recently, access to the crystal structures of ribosomes, either alone or bound to a variety of antibiotics has provided additional ways to study antibiotic action. This has led to an understanding of how extant resistance mechanisms may be circumvented by identifying new structural classes that bind in substantially different ways or at different sites on the ribosome. In addition, certain molecular inhibitors bind to and inhibit their targets in an induced-fit mode, and this has been seen with some ribosomal inhibitors. Since this type of interaction may not be immediately recognized in a structure-based design process, the discovery of inhibitors of function remains a useful method for novel drug discovery.

### SUMMARY OF THE INVENTION

In one embodiment, a method of treating a bacterial infection in a subject comprises administering to the subject who would benefit from such treatment a therapeutically effective amount of a pharmaceutically acceptable formulation comprising an antibiotic compound as described below.

In one embodiment, a method of treating a bacterial infection in a subject comprises administering to a subject who would benefit from such treatment a therapeutically effective amount of a pharmaceutically acceptable formulation comprising an antibiotic compound having the structure (I):

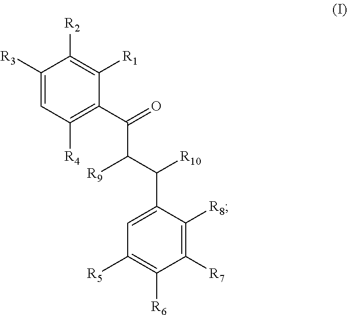

wherein: R₁ is —H, —OH, —OR₁₁, —R₁₁, —CN, —CH₂-Ph, or -4-hydroxybenzyl; R₂ is —H, —OH, —R₁₁, —CN, —CH₂-Ph, or -4-hydroxybenzyl; R₃ is —H, —OH, —OR₁₁, —R₁₁, —CN, —CH₂-Ph, or -4-hydroxybenzyl; R₄ is —H, —OH, —R₁, —CN, —CH₂-Ph, or -4-hydroxybenzyl; R₅ is —H, —OH, —OR₁₁, —R₁₁, —CN, or —CH₂—CH═C(Me)₂; R₆ is —H, —OH, —OR₁₁, —R₁₁, —CN, or —CH₂—CH═C(Me)₂; R₇ is —H, —OH, —OR₁₁, —R₁₁, —CN, or —CH₂—CH═C(Me)₂; R₈ is —H, —OH, —OR₁₁, —R₁₁, —CN, or —CH₂—CH═C(Me)₂; R₉ and R₁₀ are —H or together form an additional bond; R₁₁ is C₁-C₆ alkyl; and
wherein at least one of R₁, R₂, R₃, and R₄ is not hydrogen.

In one embodiment, a method of treating a bacterial infection in a subject comprises administering to the subject who would benefit from such treatment a therapeutically effective amount of a pharmaceutically acceptable formulation comprising an antibiotic compound having the structure (II):

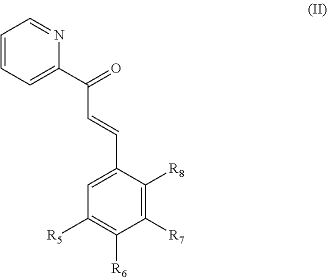

wherein: R₅ is —H, —OH, —R₁₁, —CN, or —CH₂—CH═C(Me)₂; R₆ is —H, —OH, —R₁₁, —CN, or —CH₂—CH═C(Me)₂; R₇ is —H, —OH, —OR₁₁, —R₁₁, —CN, or —CH₂—CH═C(Me)₂; R₈ is —H, —OH, —R₁₁, —CN, or —CH₂—CH═C(Me)₂; R₁₁ is C₁-C₆ alkyl; and wherein at least one of R₁, R₂, R₃, and R₄ is not hydrogen.

In one embodiment, a method of treating a bacterial infection in a subject comprises administering to the subject who would benefit from such treatment a therapeutically effective amount of a pharmaceutically acceptable formulation comprising an antibiotic compound having the structure (III):

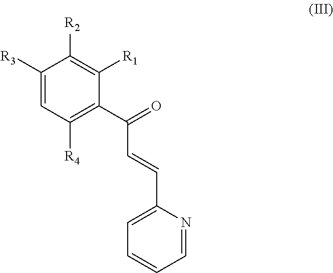

wherein: R₁ is —H, —OH, —OR₁₁, —R₁₁, —CN, —CH₂-Ph, or -4-hydroxybenzyl; R₂ is —H, —OH, —R₁₁, —CN, —CH₂-Ph, or -4-hydroxybenzyl; R₃ is —H, —OH, —OR₁₁, —R₁₁, —CN, —CH₂-Ph, or -4-hydroxybenzyl; R₄ is —H, —OH, —R₁, —CN, —CH₂-Ph, or -4-hydroxybenzyl; R₁₁ is C₁-C₆ alkyl; and wherein at least one of R₁, R₂, R₃, and R₄ is not hydrogen.

In one embodiment, a method of treating a bacterial infection in a subject comprises administering to the subject who would benefit from such treatment a therapeutically effective amount of a pharmaceutically acceptable formulation comprising an antibiotic compound having the structure (IV):

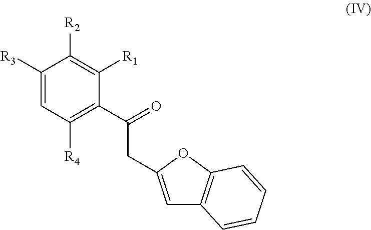

wherein: R₁ is —H, —OH, —OR₁₁, —R₁₁, —CN, —CH₂-Ph, or -4-hydroxybenzyl; R₂ is —H, —OH, —R₁₁, —CN, —CH₂-Ph, or -4-hydroxybenzyl; R₃ is —H, —OH, —OR₁₁, —R₁₁, —CN, —CH₂-Ph, or -4-hydroxybenzyl; R₄ is —H, —OH, —R₁, —CN, —CH₂-Ph, or -4-hydroxybenzyl; R₁₁ is C₁-C₆ alkyl; and wherein at least one of R₁, R₂, R₃, and R₄ is not hydrogen.

In an embodiment, a method of treating a bacterial infection in a subject comprises administering to the subject who would benefit from such treatment a therapeutically effective amount of a pharmaceutically acceptable formulation comprising an antibiotic compound having the structure (V):

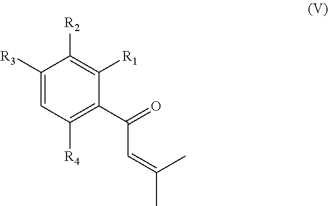

wherein: R₁ is —H, —OH, —OR₁₁, —R₁₁, —CN, —CH₂-Ph, or -4-hydroxybenzyl; R₂ is —H, —OH, —R₁₁, —CN, —CH₂-Ph, or -4-hydroxybenzyl; R₃ is —H, —OH, —OR₁₁, —R₁₁, —CN, —CH₂-Ph, or -4-hydroxybenzyl; R₄ is —H, —OH, —OR₁₁, —R₁, —CN, —CH₂-Ph, or -4-hydroxybenzyl; R₁₁ is C₁-C₆ alkyl; and wherein at least one of R₁, R₂, R₃, and R₄ is not hydrogen.

In an embodiment, a method of treating a bacterial infection in a subject comprises administering to the subject who would benefit from such treatment a therapeutically effective amount of a pharmaceutically acceptable formulation comprising an antibiotic compound having the structure (VI):

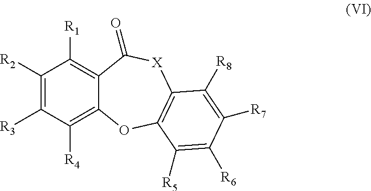

wherein: X is O, NH, or NR₉; R₁ is —H; —R₉; —Cl; —F; —OH; —OR₉; —NH₂; —NHR₉; —N(R₉)₂; —CH₂—C(O)—CH₂—CH₂—CH₂—CH₂—CH₃; —CH₂—C(O)—CH₂—CH₂—CH₂—CH₃; -(E)-C(CH₃)═CH(CH₃); —(Z)—C(CH₃)═CH(CH₃); —CH₂—OH; —CHO; —NH—C(O)—NH—R₁₀; or —NH—C(O)—R₁₀; R₂ is —H; —R₉; —Cl; —F; —OH; —OR₉; —NH₂; —NHR₉; —N(R₉)₂; —CH₂—C(O)—CH₂—CH₂—CH₂—CH₂—CH₃; —CH₂—C(O)—CH₂—CH₂—CH₂—CH₃; -(E)-C(CH₃)═CH(CH₃); —(Z)—C(CH₃)═CH(CH₃); —CH₂—OH; —CHO; —NH—C(O)—NH—R₁₀; or —NH—C(O)—R₁₀; R₃ is —H; —R₉; —Cl; —F; —OH; —OR₉; —NH₂; —NHR₉; —N(R₉)₂; —CH₂—C(O)—CH₂—CH₂—CH₂—CH₂—CH₃; —CH₂—C(O)—CH₂—CH₂—CH₂—CH₃; -(E)-C(CH₃)═CH(CH₃); —(Z)—C(CH₃)═CH(CH₃); —CH₂—OH; —CHO; —NH—C(O)—NH—R₁₀; or —NH—C(O)—R₁₀; R₄ is —H; —R₉; —Cl; —F; —OH; —OR₉; —NH₂; —NHR₉; —N(R₉)₂; —CH₂—C(O)—CH₂—CH₂—CH₂—CH₂—CH₃; —CH₂—C(O)—CH₂—CH₂—CH₂—CH₃; -(E)-C(CH₃)═CH(CH₃); —(Z)—C(CH₃)═CH(CH₃); —CH₂—OH; —CHO; —NH—C(O)—NH—R₁₀; or —NH—C(O)—R₁₀; R₅ is —H; —R₉; —Cl; —F; —OH; —OR₉; —NH₂; —NHR₉; —N(R₉)₂; -(E)-C(CH₃)═CH(CH₃); (Z)—C(CH₃)═CH(C_{H3}); —CO₂H; —CO₂⁻; —CO₂R₉; —CH₂—OH; —CH₂—CH═C(CH₃)₂; —CHO; or —CH₂—C(O)—CH₃; R₆ is —H; —R₉; —Cl; —F; —OH; —OR₉; —NH₂; —NHR₉; —N(R₉)₂; -(E)-C(CH₃)CH(CH₃); (Z)—C(CH₃)═CH(CH₃); —CO₂H; —CO₂⁻; —CO₂R₉; —CH₂—OH; —CH₂—CH═C(CH₃)₂; —CHO; or —CH₂—C(O)—CH₃; R₇ is —H; —R₉; —Cl; —F; —OH; —OR₉; —NH₂; —NHR₉; —N(R₉)₂; -(E)-C(CH₃)CH(CH₃); (Z)—C(CH₃)═CH(CH₃); —CO₂H; —CO₂⁻; —CO₂R₉; —CH₂—OH; —CH₂—CH═C(CH₃)₂; —CHO; or —CH₂—C(O)—CH₃; R₈ is —H; —R₉; —Cl; —F; —OH; —OR₉; —NH₂; —NHR₉; —N(R₉)₂; -(E)-C(CH₃)CH(CH₃); (Z)—C(CH₃)═CH(CH₃); —CO₂H; —CO₂⁻; —CO₂R₉; —CH₂—OH; —CH₂—CH═C(CH₃)₂; —CHO; or —CH₂—C(O)—CH₃; R₉ is C₁-C₆ alkyl; R₁₀ is benzene or naphthalene substituted with one or more of the following: —H; —R₉; —Cl; —F; —OH; —OR₉; —NH₂; —NHR₉; —N(R₉)₂; and wherein at least one of R₁, R₂, R₃, R₄, R₅, R₆, R₇, and R₈ is not hydrogen.

In one embodiment, a method of treating a bacterial infection in a subject comprising administering to the subject who would benefit from such treatment a therapeutically effective amount of a pharmaceutically acceptable formulation comprising an antibiotic compound having the structure (VII):

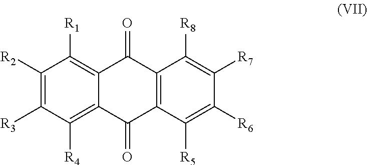

wherein each of R₁-R₈ is independently: —H; —R₉; —Cl; —F; —OH; —OR₉; —NH₂; —NHR₉; —N(R₉)₂; —CH₂—OH; or —CHO; and wherein at least one of R₁, R₂, R₃, R₄, R₅, R₆, R₇, and R₈ is not hydrogen.

In one embodiment, a method of treating a bacterial infection in a subject comprises administering to the subject who would benefit from such treatment a therapeutically effective amount of a pharmaceutically acceptable formulation comprising an antibiotic compound having the structure (VIII):

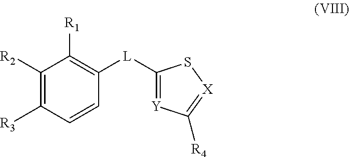

wherein: L is: —NH—C(O)—NH—; —C(O)—NH—; —C(CH₃)—NH—C(O)—NH—; —CH₂—C(O)—NH—; —CH₂—CH(OH)—CH₂—NH—C(O)—CH₂—; X is: N; CH; CMe; C—CO₂H; C—CO₂R; C—CONH₂; C—CONHR₅; or C—CON(R₅)₂; Y is: N; CH; CMe; C—CO₂H; or C—CO₂R; C—CONH₂; C—CONHR₅; or C—CON(R₅)₂; R₁ is: —H; —CF₃; —R₅; —Cl; —F; —OH; or —OR₅; R₂ is: —H; —CF₃; —R₅; —Cl; —F; —OH; or —OR₅; R₃ is: —H; —CF₃; —R₅; —Cl; —F; —OH; or —OR₅; R₄ is: —R₅; —S—R₅; —S—CH₂—CH═CH₂; —S—CH₂—C≡CH; phenyl; or furanyl; R₅ is C₁-C₆ alkyl; and wherein at least one of R₁, R₂, R₃, and R₄ is not hydrogen.

In one embodiment, a method of treating a bacterial infection in a subject comprises administering to the subject who would benefit from such treatment a therapeutically effective amount of a pharmaceutically acceptable formulation comprising an antibiotic compound having the structure (IX):

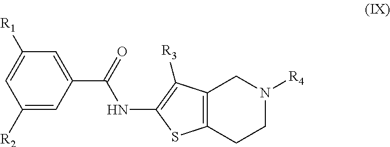

R₁ is: —H; —CF₃; —R₅; —Cl; —F; —OH; or —OR₅; R₂ is: —H; —CF₃; —R₅; —Cl; —F; —OH; or —OR₅; R₃ is —H; —CO₂H; —CO₂R₅; —CONH₂; —CONR₅; or —CON(R₅)₂; and R₄ is —H or —R₅; R₅ is C₁-C₆ alkyl; and wherein at least one of R₁, R₂, R₃, and R₄ is not hydrogen.

In one embodiment, a method of treating a bacterial infection in a subject comprises administering to the subject who would benefit from such treatment a therapeutically effective amount of a pharmaceutically acceptable formulation comprising an antibiotic compound having the structure (XI):

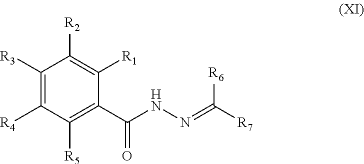

wherein: R₁ is: —H; —NO₂; —F; —Cl; —Br; OH; —OR₈; or —CF₃; R₂ is: —H; —NO₂; —F; —Cl; —Br; OH; —OR₈; or —CF₃; R₃ is: —H; —NO₂; —F; —Cl; —Br; OH; —OR₈; or —CF₃; or R₂ and R₃ combine to form an aromatic ring; R₄ is: —H; —NO₂; —F; —Cl; —Br; OH; —OR₈; or —CF₃; R₅ is: —H; —NO₂; —F; —Cl; —Br; OH; —OR₈; or —CF₃; wherein at least one of R₁, R₂, R₃, R₄, and R₅ is not hydrogen; R₆ is: —H or C₁-C₆ alkyl; R₇ is an aryl or heterocyclic substituent; and R₈ is C₁-C₆ alkyl.

In one embodiment, a method of treating a bacterial infection in a subject comprises administering to the subject who would benefit from such treatment a therapeutically effective amount of a pharmaceutically acceptable formulation comprising an antibiotic compound having the structure (XII):

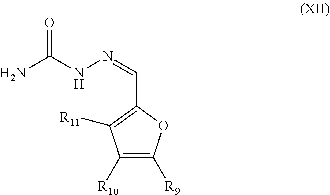

wherein each R₉, R₁₀, and R₁₁ are independently: —H; —NO₂; —NH₂; —NH(R₁₆); —N(R₁₆)₂; —F; —Cl; —Br; —OH; —OR₈; or —CF₃; wherein at least one of R₉, R₁₀, and R₁₁ is not hydrogen; and wherein R₁₆ is C₁-C₆ alkyl.

In one embodiment, a method of treating a bacterial infection in a subject comprises administering to the subject who would benefit from such treatment a therapeutically effective amount of a pharmaceutically acceptable formulation comprising an antibiotic compound having the structure (XIII):

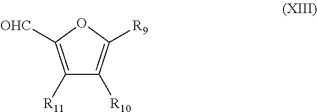

where each R₉, R₁₀, and R₁₁ are independently: —H; —NO₂; —NH₂; —NH(R₁₆); —N(R₁₆)₂; —F; —Cl; —Br; —OH; —OR₈; or —CF₃; wherein at least one of R₉, R₁₀, and R₁₁ is not hydrogen; and wherein R₁₆ is C₁-C₆ alkyl.

In one embodiment, a method of treating a bacterial infection in a subject comprises administering to the subject who would benefit from such treatment a therapeutically effective amount of a pharmaceutically acceptable formulation comprising an antibiotic compound having the structure (XIV):

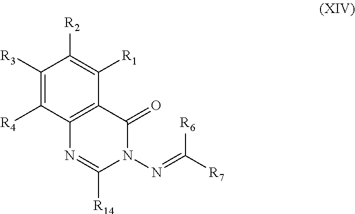

wherein: R₁ is: —H; —NO₂; —F; —Cl; —Br; OH; —OR₈; or —CF₃; R₂ is: —H; —NO₂; —F; —Cl; —Br; OH; —OR₈; or —CF₃; R₃ is: —H; —NO₂; —F; —Cl; —Br; OH; —OR₈; or —CF₃; or R₂ and R₃ combine to form an aromatic ring; R₄ is: —H; —NO₂; —F; —Cl; —Br; OH; —OR₈; or —CF₃; wherein at least one of R₁, R₂, R₃, and R₄ is not hydrogen; R₆ is: —H or C₁-C₆ alkyl; R₇ is an aryl or heterocyclic substituent; and R₁₄ is C₁-C₆ alkyl.

In one embodiment, a method of treating a bacterial infection in a subject comprises administering to the subject who would benefit from such treatment a therapeutically effective amount of a pharmaceutically acceptable formulation comprising an antibiotic compound having the structure (XV):

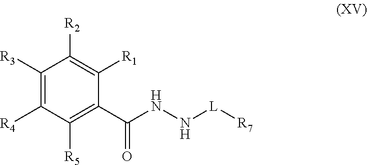

wherein: R₁ is: —H; —NO₂; —F; —Cl; —Br; OH; —OR₈; or —CF₃; R₂ is: —H; —NO₂; —F; —Cl; —Br; OH; —OR₈; or —CF₃; R₃ is: —H; —NO₂; —F; —Cl; —Br; OH; —OR₈; or —CF₃; or R₂ and R₃ combine to form an aromatic ring; R₄ is: —H; —NO₂; —F; —Cl; —Br; OH; —OR₈; or —CF₃; R₅ is: —H; —NO₂; —F; —Cl; —Br; OH; —OR₈; or —CF₃; wherein at least one of R₁, R₂, R₃, R₄, and R₅ is not hydrogen; R₇ is alkyl, alkenyl, aryl or a heterocyclic substituent; R₈ is C₁-C₆ alkyl; and where L is: —C(O)—NH—CH₂—; —C(O)—NH—CH₂—CH₂—; —C(O)—CH₂—O—; ═CH—CH═CH—; —C(O)—; CH—; or ═C(Me)-.

In one embodiment, a method of treating a bacterial infection in a subject comprises administering to the subject who would benefit from such treatment a therapeutically effective amount of a pharmaceutically acceptable formulation comprising an antibiotic compound having the structure (XVI):

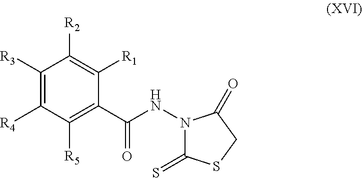

wherein: R₁ is: —H; —NO₂; —F; —Cl; —Br; OH; —OR₈; or —CF₃; R₂ is: —H; —NO₂; —F; —Cl; —Br; OH; —OR₈; or —CF₃; R₃ is: —H; —NO₂; —F; —Cl; —Br; OH; —OR₈; or —CF₃; or R₂ and R₃ combine to form an aromatic ring; R₄ is: —H; —NO₂; —F; —Cl; —Br; OH; —OR₈; or —CF₃; R₅ is: —H; —NO₂; —F; —Cl; —Br; OH; —OR₈; or —CF₃; wherein at least one of R₁, R₂, R₃, R₄, and R₅ is not hydrogen; and R₈ is C₁-C₆ alkyl.

In one embodiment, a method of treating a bacterial infection in a subject comprises administering to the subject who would benefit from such treatment a therapeutically effective amount of a pharmaceutically acceptable formulation comprising an antibiotic compound having the structure (XVII):

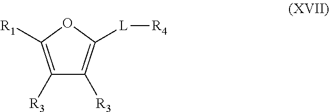

wherein: R₁ is: —H; —NO₂; —F; —Cl; —Br; OH; —OR₈; or —CF₃; R₂ is: —H; —NO₂; —F; —Cl; —Br; OH; —OR₈; or —CF₃; R₃ is: —H; —NO₂; —F; —Cl; —Br; OH; —OR₈; or —CF₃; R₄ is aryl or a substituted or unsubstituted heterocyclic substituent; R₈ is C₁-C₆ alkyl; and wherein L is: -(E)-CH═CH—C(N—N═C(NH₂)₂)-(E)-CH═CH—; -(E)-CH═CH—C(N—N═C(NH₂)₂)—(Z)—CH═CH—; —(Z)—CH═CH—C(N—N═C(NH₂)₂)-(E)-CH═CH—; —(Z)—CH═CH—C(N—N═C(NH₂)₂)—(X)—CH═CH—; -(E)-CH═CH—C(O)-(E)-CH═CH—; -(E)-CH═CH—C(O)—(Z)—CH═CH—; —(Z)—CH═CH—C(O)-(E)-CH═CH—; —(Z)—CH═CH—C(O)—(Z)—CH═CH—; -(E)-CH═CH—CH═N—NH—C(S)—NH—; —(Z)—CH═CH—CH═N—NH—C(S)—NH—; -(E)-CH CH—; —(Z)—CH═CH—; -(E)-CH═CH—C(Me)N—N═CH—; —(Z)—CH═CH—C(Me)N—N═CH—;

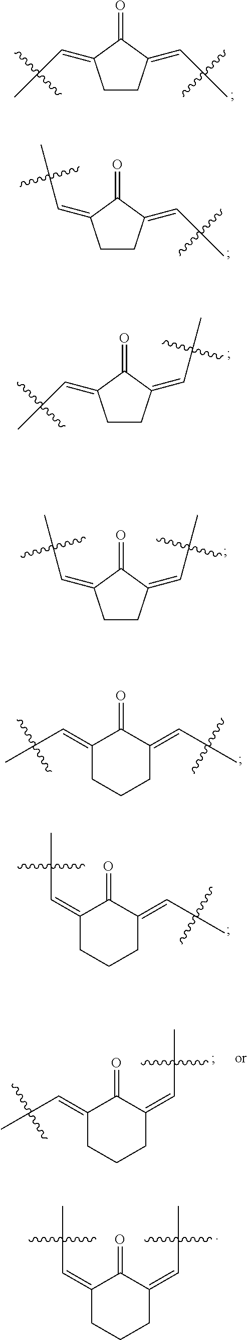

In one embodiment, a method of treating a bacterial infection in a subject comprises administering to the subject who would benefit from such treatment a therapeutically effective amount of a pharmaceutically acceptable formulation comprising an antibiotic compound having the structure (XVIII):

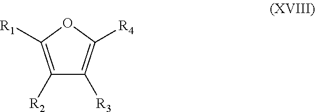

wherein: R₁ is: —H; —NO₂; —F; —Cl; —Br; OH; —OR₈; or —CF₃; R₂ is: —H; —NO₂; —F; —Cl; —Br; OH; —OR₈; or —CF₃; R₃ is: —H; —NO₂; —F; —Cl; —Br; OH; —OR₈; or —CF₃; R₄ is: -(E)-CH═CH—C(N—N═C(NH₂)₂)-(E)-CH═CH₂; —R—CH═CH—C(N—N═C(NH₂)₂)—(Z)—CH═CH₂; —(Z)—CH═CH—C(N—N═C(NH₂)₂)—R—CH═CH₂; —(Z)—CH═CH—C(N—N═C(NH₂)₂)—(X)—CH═CH₂; -(E)-CH═CH—C(O)-(E)-CH═CH₂; —R—CH═CH—C(O)—(Z)—CH═CH₂; —(Z)—CH═CH—C(O)-(E)-CH═CH₂; —(Z)—CH═CH—C(O)—(Z)—CH═CH₂; -(E)-CH═CH—CH═N—NH—C(S)—NH₂; —(Z)—CH═CH—CH═N—NH—C(S)—NH₂; -(E)-CH═CH₂—R₇; —(Z)—CH═CH₂—R₇; —R—CH═CH—C(Me)N—N═CH₂; —(Z)—CH═CH—C(Me)N—N═CH₂; —R—CH═CH—C(Me)=N—NH—C(NH)—NH₂; —(Z)—CH═CH—C(Me)N—NH—C(NH)—NH₂;

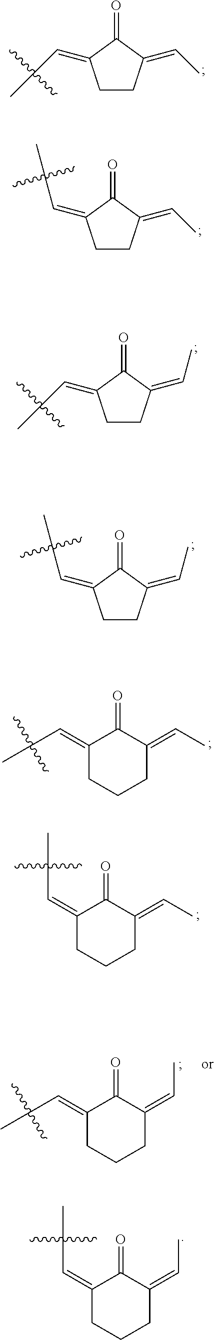

In one embodiment, a method of treating a bacterial infection in a subject comprises administering to the subject who would benefit from such treatment a therapeutically effective amount of a pharmaceutically acceptable formulation comprising an antibiotic compound having the structure (XIX):

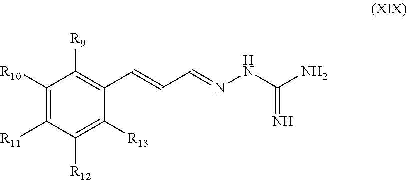

wherein each R₉, R₁₀, R₁₁, R₁₂, and R₁₃ are independently: —H; —NO₂; —F; —Cl; —Br; OH; —OR₈; or —CF₃; and wherein at least one of R₉, R₁₀, R₁₁, R₁₂, and R₁₃ is not hydrogen.

In one embodiment, a method of treating a bacterial infection in a subject comprises administering to the subject who would benefit from such treatment a therapeutically effective amount of a pharmaceutically acceptable formulation comprising an antibiotic compound having the structure (XX):

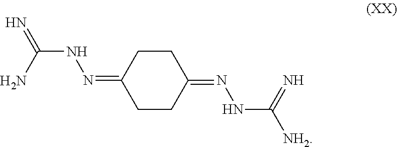 [SMILES: N=C(N)NN=C1CCC(=NNC(=N)N)CC1]

In one embodiment, a method of treating a bacterial infection in a subject comprises administering to the subject who would benefit from such treatment a therapeutically effective amount of a pharmaceutically acceptable formulation comprising an antibiotic compound having the structure (XXI):

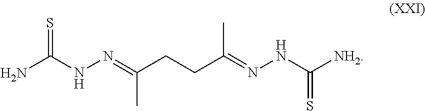 [SMILES: C/C(CC/C(C)=N/NC(N)=S)=N\NC(N)=S]

In one embodiment, a method of treating a bacterial infection in a subject comprises administering to the subject who would benefit from such treatment a therapeutically effective amount of a pharmaceutically acceptable formulation comprising an antibiotic compound having the structure (XXII):

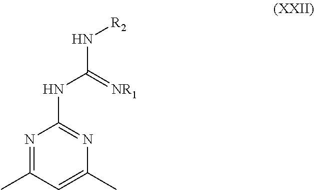

wherein: R₁ is —H; —Ar; —SO₂Ar;
R₂ is: —OH; —Ar; —C(O)—CHR₃—Ar;

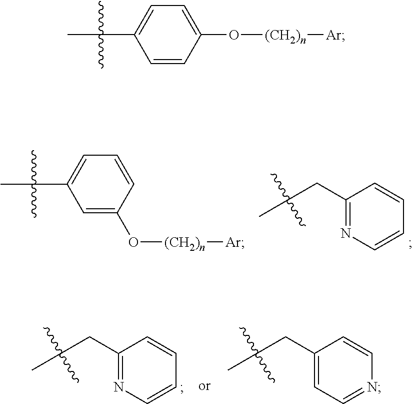

and
wherein n is 0 or 1.

In one embodiment, a method of treating a bacterial infection in a subject comprising administering to the subject who would benefit from such treatment a therapeutically effective amount of a pharmaceutically acceptable formulation comprising an antibiotic compound having the structure (XXIII):

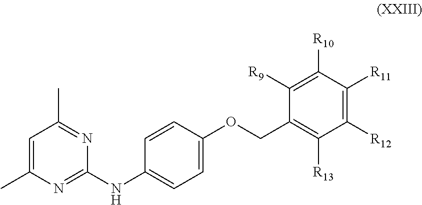

wherein each of R₉, R₁₀, R₁₁, R₁₂, and R₁₃ are independently: —H; —NO₂; —F; —Cl; —Br; OH; —OR₈; —OBn; or —CF₃; where Bn is benzyl; R₈ is C₁-C₆ alkyl; and at least one of R₉, R₁₀, R₁₁, R₁₂, and R₁₃ is not hydrogen.

In one embodiment, a method of treating a bacterial infection in a subject comprising administering to the subject who would benefit from such treatment a therapeutically effective amount of a pharmaceutically acceptable formulation comprising an antibiotic compound having the structure (XXIV):

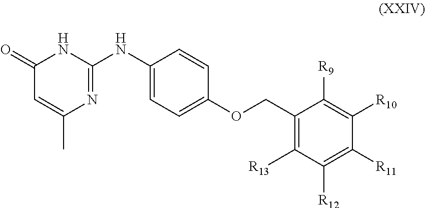

wherein each of R₉, R₁₀, R₁₁, R₁₂, and R₁₃ are independently: —H; —NO₂; —F; —Cl; —Br; OH; —OR₈; —OBn; or —CF₃; where Bn is benzyl; R₈ is C₁-C₆ alkyl; and at least one of R₉, R₁₀, R₁₁, R₁₂, and R₁₃ is not hydrogen.

In one embodiment, a method of treating a bacterial infection in a subject comprising administering to the subject who would benefit from such treatment a therapeutically effective amount of a pharmaceutically acceptable formulation comprising an antibiotic compound having the structure (XXV):

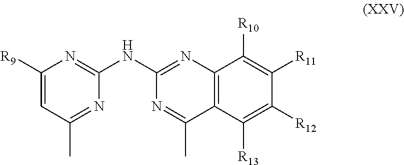

wherein: R₉ is —H or -Me; each of R₁₀, R₁₁, R₁₂, and R₁₃ are independently: —H; —NO₂; —F; —Cl; —Br; OH; —OR₈; —OBn; or —CF₃; where Bn is benzyl; R₈ is C₁-C₆ alkyl; and at least one of R₉, R₁₀, R₁₁, R₁₂, and R₁₃ is not hydrogen.

In one embodiment, a method of treating a bacterial infection in a subject comprising administering to the subject who would benefit from such treatment a therapeutically effective amount of a pharmaceutically acceptable formulation comprising an antibiotic compound having the structure (XXVI):

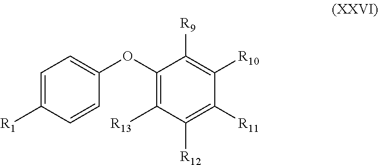

wherein: R₁ is —NH₂; —NHR₈; —N(R₈)₂; —NH—C(O)—NH₂; —NH—C(NH)—NH₂; —NH—C(NR₈)—NH₂; —NH—C(S)—NH₂; each of R₉, R₁₀, R₁₁, R₁₂, and R₁₃ are independently: —H; —NO₂; —F; —Cl; —Br; OH; —OR₈; —OBn; or —CF₃; where Bn is benzyl; R₈ is C₁-C₆ alkyl; and at least one of R₉, R₁₀, R₁₁, R₁₂, and R₁₃ is not hydrogen.

In one embodiment, a method of treating a bacterial infection in a subject comprising administering to the subject who would benefit from such treatment a therapeutically effective amount of a pharmaceutically acceptable formulation comprising an antibiotic compound having the structure (XXVII):

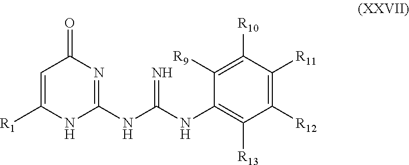

wherein: R₁ is —H; —C₁-C₆ alkyl; or —CH₂—R₂; R₂ is —OH; —OR₈; —CO₂H; —CO₂R₈; each of R₉, R₁₀, R₁₁, R₁₂, and R₁₃ are independently: —H; —NO₂; —F; —Cl; —Br; OH; —OR₈; —OBn; or —CF₃; where Bn is benzyl; R₈ is C₁-C₆ alkyl; and at least one of R₉, R₁₀, R₁₁, R₁₂, and R₁₃ is not hydrogen.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts graphs showing the activity (% pos) vs. concentration for each representative compound of each class in an aminoacylation/translation (A/T) assay;

FIG. 2 depicts graphs showing the activity (% pos) vs. concentration for each representative compound of Classes 1-4 in a P. aeruginosa PheRS aminoacylation assay;

FIG. 3 depicts the data collected from time-kill experiments to determine the efficacy against both Gram (+) and a Gram (−) organisms;

FIG. 4 depicts data collected from tests of compounds from the six classes against the protein synthesis systems from eukaryotic origins (wheat germ cell extracts); and

FIG. 5 depicts data collected from tests of compounds from classes 1-4 as inhibitors of P. aeruginosa PheRS on the activity of human mitochondrial PheRS (hmPheRS) using an aminoacylation assay containing hmPheRS.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood that the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include”, and derivations thereof, mean “including, but not limited to.” The term “coupled” means directly or indirectly connected.

Compounds described herein embrace both racemic and optically active compounds. Chemical structures depicted herein that do not designate specific stereochemistry are intended to embrace all possible stereochemistries.

Compounds described herein embrace isomer mixtures, racemic, optically active, and optically inactive stereoisomers and compounds.

### DEFINITIONS

Unless defined otherwise, all technical and scientific terms used herein have the same meaning as commonly understood by one of ordinary skill in the art.

The term “acyl,” as used herein, generally refers to a carbonyl substituent, —C(O)R, where R is alkyl or substituted alkyl, aryl, or substituted aryl, which may be called an alkanoyl substituent when R is alkyl.

The terms “administration,” “administering,” or the like, as used herein, when used in the context of providing a pharmaceutical composition to a subject generally refers to providing to the subject one or more pharmaceutical or “over-the-counter” (OTC) compositions in combination with an appropriate delivery vehicle by any means such that the administered compound achieves one or more of the intended biological effects for which the compound was intended. By way of non-limiting example, a composition may be administered by parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or disease state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

The terms “alkenyl” and “olefin,” as used herein, generally refer to any structure or moiety having the unsaturation C═C.

As used herein, the term “alkynyl” generally refers to any structure or moiety having the unsaturation C≡C.

The term “alkoxy,” as used herein, generally refers to an —OR group, where R is an alkyl, unsubstituted or substituted lower alkyl (C₁-C₆), aryl, substituted aryl. Alkoxy groups include, for example, methoxy, ethoxy, phenoxy, substituted phenoxy, benzyloxy, phenethyloxy, t-butoxy, and others.

The term “alkyl,” as used herein, generally refers to a chemical substituent containing the monovalent group CₙH₂ₙ, where n is an integer greater than zero. Alkyl includes a branched or unbranched monovalent hydrocarbon radical. An “Cₙ-Cₘ” alkyl or refers to all alkyl groups (branched or unbranched) containing from n to m carbon atoms. For example, a C₁-C₆ alkyl refers to a methyl, ethyl, propyl, butyl, pentyl, and hexyl, as well as branched alkyl groups containing up to 6 carbons. All possible isomers of an indicated alkyl are also included. Thus, propyl includes isopropyl, butyl includes n-butyl, isobutyl and t-butyl, and so on. The term alkyl includes substituted alkyls. For example, alkyl includes, but is not limited to: methyl, ethyl, propyl, isopropyl, butyl, iso-butyl, sec-butyl, pentyl, 3-pentyl, hexyl, heptyl, octyl, nonyl, decyl, undecyl, dodecyl, tridecyl, tetradecyl or pentadecyl; “alkenyl” includes but is not limited to vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl; 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, 1-tridecenyl, 2-tridecenyl, 3-tridecenyl, 4-tridecenyl, 5-tridecenyl, 6-tridecenyl, 7-tridecenyl, 8-tridecenyl, 9-tridecenyl, 10-tridecenyl, 11-tridecenyl, 12-tridecenyl, 1-tetradecenyl, 2-tetradecenyl, 3-tetradecenyl, 4-tetradecenyl, 5-tetradecenyl, 6-tetradecenyl, 7-tetradecenyl, 8-tetradecenyl, 9-tetradecenyl, 10-tetradecenyl, 11-tetradecenyl, 12-tetradecenyl, 13-tetradeceny, 1-pentadecenyl, 2-pentadecenyl, 3-pentadecenyl, 4-pentadecenyl, 5-pentadecenyl, 6-pentadecenyl, 7-pentadecenyl, 8-pentadecenyl, 9-pentadecenyl, 10-pentadecenyl, 11-pentadecenyl, 12-pentadecenyl, 13-pentadecenyl, 14-pentadecenyl; “alkoxy” includes but is not limited to methoxy, ethoxy, propoxy, isopropoxy, butoxy, iso-butoxy, sec-butoxy, pentoxy, 3-pentoxy, hexoxy, heptyloxy, octyloxy, nonyloxy, decyloxy, undecyloxy, dodecyloxy, tridecyloxy, tetradecyloxy, or pentadecyloxy.

The term “amino,” as used herein, generally refers to a group —NRR′, where R and R′ may independently be hydrogen, lower alkyl, substituted lower alkyl, aryl, substituted aryl or acyl.

The term “analog,” as used herein, generally refers to a compound that resembles another in structure but is not necessarily an isomer.

The term “aryl,” as used herein, generally refers to a chemical substituent containing an aromatic group. An aromatic group may be a single aromatic ring or multiple aromatic rings that are fused together, coupled covalently, or coupled to a common group such as a methylene, ethylene, or carbonyl, and includes polynuclear ring structures. An aromatic ring or rings may include, but is not limited to, substituted or unsubstituted phenyl, naphthyl, biphenyl, diphenylmethyl, and benzophenone groups. The term “aryl” includes substituted aryls.

“Derivative” in the context of this application is generally defined as a chemical substance derived from another substance either directly or by modification or partial substitution.

“Analog” in the context of this application is generally defined as a compound that resembles another in structure but is not necessarily an isomer. Typical analogs or derivatives include molecules which demonstrate equivalent or improved biologically useful and relevant function, but which differ structurally from the parent compounds. Such analogs or derivatives may include, but are not limited to, esters, ethers, carbonates, amides, carbamates, phosphate esters and ethers, sulfates, glycoside ethers, with or without spacers (linkers).

The terms “coupling” and “coupled,” as used herein, with respect to molecular moieties or species, atoms, synthons, cyclic compounds, and nanoparticles refers to their attachment or association with other molecular moieties or species, atoms, synthons, cyclic compounds, and nanoparticles. The attachment or association may be specific or non-specific, reversible or non-reversible, the result of chemical reaction, or complexation or charge transfer. The bonds formed by a coupling reaction are often covalent bonds, or polar-covalent bonds, or mixed ionic-covalent bonds, and may sometimes be Coulombic forces, ionic or electrostatic forces or interactions.

The term “cycloalkyl,” as used herein, includes, but is not limited to, cyclopropyl, cyclobutyl, cyclopentyl, cyclohexyl, cycloheptyl, or cyclooctyl.

The term “functionalized,” as used herein, generally refers to the presence of a reactive chemical moiety or functionality. A functional group may include, but is not limited to, chemical groups, biochemical groups, organic groups, inorganic groups, organometallic groups, aryl groups, heteroaryl groups, cyclic hydrocarbon groups, amino (—NH2), hydroxyl (—OH), cyano (—C≡N), nitro (NO₂), carboxyl (—COOH), formyl (—CHO), keto (—CH₂C(O)CH₂—), ether (—CH₂—O—CH₂—), thioether (—CH₂—S—CH₂—), alkenyl (—C═C—), alkynyl, (—C≡C—), epoxy (e.g.,), metalloids (functionality containing Si and/or B) and halo (F, Cl, Br, and I) groups. In some embodiments, the functional group is an organic group.

The term “heteroaryl,” as used herein, generally refers to a completely unsaturated heterocycle.

The term “heterocycle,” as used herein, generally refers to a closed-ring structure, in which one or more of the atoms in the ring is an element other than carbon. Heterocycle may include aromatic compounds or non-aromatic compounds. Heterocycles may include rings such as thiophene, pyridine, isoxazole, phthalimide, pyrazole, indole, furan, or benzo-fused analogs of these rings. Examples of heterocycles include tetrahydrofuran, furan, pyrrole, morpholine, piperidine, pyrrolidine, and others. In some embodiments, “heterocycle” is intended to mean a stable 5- to 7-membered monocyclic or 7- to 10-membered bicyclic heterocyclic ring which is either saturated or unsaturated, and which consists of carbon atoms and from 1 to 4 heteroatoms (e.g., N, O, and S) and wherein the nitrogen and sulfur heteroatoms may optionally be oxidized, and the nitrogen may optionally be quaternized, and including any bicyclic group in which any of the above-defined heterocyclic rings is fused to a benzene ring. In some embodiments, heterocycles may include cyclic rings including boron atoms. The heterocyclic ring may be attached to its pendant group at any heteroatom or carbon atom that results in a stable structure. The heterocyclic rings described herein may be substituted on carbon or on a nitrogen atom if the resulting compound is stable. Examples of such heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzofuranyl, benzothiophenyl, carbazole, chromanyl, chromenyl, cinnolinyl, decahydroquinolinyl, furanyl, furazanyl, imidazolidinyl, imidazolinyl, imidazolyl, indolinyl, indolizinyl, indolyl, isobenzofuranyl, isochromanyl, isoindolinyl, isoindolyl, isoquinolinyl (benzimidazolyl), isothiazolyl, isoxazolyl, morpholinyl, naphthyridinyl, octahydroisoquinolinyl, oxazolidinyl, oxazolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridinyl, pyridyl, pyrimidinyl, pyrrolidinyl, pyrrolinyl, pyrrolyl, quinazolinyl, quinolinyl, quinoxalinyl, quinuclidinyl, carbolinyl, tetrahydrofuranyl, tetrahydroisoquinolinyl, tetrahydroquinolinyl, tetrazolyl, thianthrenyl, thiazolyl, thienyl, thiophenyl, triazinyl, xanthenyl. Also included are fused ring and spiro compounds containing, for example, the above heterocycles.

The terms “in need of treatment” or “in need thereof,” as used herein, when used in the context of a subject being administered a pharmacologically active composition, generally refers to a judgment made by an appropriate healthcare provider that an individual or animal requires or will benefit from a specified treatment or medical intervention. Such judgments may be made based on a variety of factors that are in the realm of expertise of healthcare providers, but include knowledge that the individual or animal is ill, will be ill, or is at risk of becoming ill, as the result of a condition that may be ameliorated or treated with the specified medical intervention.

Terms such as “pharmaceutical composition,” “pharmaceutical formulation,” “pharmaceutical preparation,” or the like, as used herein, generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, included within the meaning of the term are pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

The term “pharmaceutically acceptable salts,” as used herein, includes salts prepared from by reacting pharmaceutically acceptable non-toxic bases or acids, including inorganic or organic bases, with inorganic or organic acids. Pharmaceutically acceptable salts may include salts derived from inorganic bases include aluminum, ammonium, calcium, copper, ferric, ferrous, lithium, magnesium, manganic salts, manganous, potassium, sodium, zinc, etc. Examples include the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, etc.

The terms “pharmaceutically acceptable formulation,” as used herein, generally refers to a non-toxic formulation containing a predetermined dosage of a pharmaceutical composition, wherein the dosage of the pharmaceutical composition is adequate to achieve a desired biological outcome. The meaning of the term may generally include an appropriate delivery vehicle that is suitable for properly delivering the pharmaceutical composition in order to achieve the desired biological outcome.

The term “pharmacologically inert,” as used herein, generally refers to a compound, additive, binder, vehicle, and the like, that is substantially free of any pharmacologic or “drug-like” activity.

The terms “Rn,” as used herein, in a chemical formula refer to hydrogen or a functional group, each independently selected, unless stated otherwise.

The term “subject,” as used herein, may be generally defined as all mammals, in particular humans.

The term “substituted heterocycle,” as used herein, generally refers to a heterocyclic group with an additional group or groups attached to any element of the heterocyclic group. Additional groups may include one or more functional groups such as lower alkyl, aryl, acyl, halogen, alkylhalos, hydroxy, amino, alkoxy, alkylamino, acylamino, acyloxy, aryloxy, aryloxyalkyl, nitro, thioether, heterocycles, both saturated and unsaturated cyclic hydrocarbons which are fused to the heterocyclic ring(s), coupled covalently or coupled to a common group such as a methylene or ethylene group, or a carbonyl coupling group such as in cyclohexyl phenyl ketone, and others.

The phrase “therapeutically effective amount,” as used herein, generally refers to an amount of a drug or pharmaceutical composition that will elicit at least one desired biological or physiological response of a cell, a tissue, a system, animal or human that is being sought by a researcher, veterinarian, physician or other caregiver.

It will be appreciated by those skilled in the art that compounds having one or more chiral center(s) may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound. As used herein, the term “single stereoisomer” refers to a compound having one or more chiral centers that, while it can exist as two or more stereoisomers, are isolated in greater than about 95% excess of one of the possible stereoisomers. As used herein a compound that has one or more chiral centers is considered to be “optically active” when isolated or used as a single stereoisomer.

### Antibiotic Screening Assays

The ribosome is a well-established target for drug discovery, but other components that are essential for protein synthesis also offer attractive targets. Elongation factor Tu (EF-Tu) delivers the charged tRNA to the A-site of the ribosome in a ternary complex with GTP and an aminoacylated tRNA, hydrolyzing the GTP to GDP in the process. Elongation factor Ts (EF-Ts) then interacts with EF-Tu to regenerate EF-Tu to an active form, facilitating the replacement of bound GDP with GTP. Elongation factor G (EF-G) plays a central role in the elongation phase of protein synthesis by catalyzing GTP-dependent translocation. EF-G is also one of the proteins involved in the termination of protein synthesis in a GTP-dependent fashion Amino-acyl tRNA synthetases (aaRS) catalyze the attachment of amino acids to their cognate tRNAs. They are essential components in protein synthesis and individually provide attractive targets for the discovery of antibiotics.

Attempts have been made to screen chemical-compound libraries by using cell extracts containing native transcription and translation systems from Escherichia coli, Streptococcus pneumoniae, and Staphylococcus aureus. This approach had only limited success. The use of cell extracts for screening can be problematic due to the presence of nucleases, degraded nucleic acids, soluble but denatured proteins, and turbidity. In addition, different preparations of S30 fractions can differ in activity and are therefore undependable. To avoid these problems, we previously developed a poly(U)-directed aminoacylation/translation (A/T) protein synthesis system which was composed of phenylalanyl-tRNA synthetases (PheRS), ribosomes, and ribosomal factors from E. coli. Using this system as a platform for screening, compounds capable of inhibiting protein synthesis in vitro and in whole-cell assays were discovered.

An aminoacylation/translation (A/T) system that contained the components required for the translation of poly(U) mRNA—ribosomes, EF-Tu, EF-Ts, EF-G, and PheRS was previously developed. Details regarding the development of assays for screening compounds may be found in the paper to Ribble et al. “Discovery and Analysis of 4H-Pyridopyrimidines, a Class of Selective Bacterial Protein Synthesis Inhibitors” Antimicrob. Agents Chemother. 2010, 54(11):4648, which is incorporated herein by reference. Below, we describe similar sets of experiments but using purified components from Pseudomonas aeruginosa.

### Aminoacylation/Translation Assays

A scintillation proximity assay (SPA) was developed using P. aeruginosa components for the aminoacylation/translation (A/T) assay as described in Ribble et al. The complete assay (50 μl) contained 50 mM Tris-HCl (pH 7.5), 25 mM KCl, 10 mM MgCl₂, 0.03 mM spermine, 1.5 mM ATP, 0.5 mM GTP, 40 μM [³H]Phenylalanine (Phe) (75 cpm/pmol) and 0.3 mg/ml polyU mRNA. To maintain constant levels of ATP and GTP the assay contained a nucleotide regeneration system composed of 4 mM phosphoenolpyruvate (PEP) and 0.025 Units/μl pyruvate kinase (PK). The concentration of ribosomes and proteins in the assay were as follows: ribosome (0.2 μM), PheRS (0.1 μM), EF-Tu (1 μM), EF-Ts (0.05 μM) and EF-G (0.2 μM). These concentrations were arrived at through sequential rounds of optimization: each concentration represents the concentration just below the saturation point for each component of the reaction in the titration.

The screening reactions were carried out in 96 well microtiter plates (Costar). Test compounds were equilibrated by addition of 33 μl of the protein/substrate mix (without tRNA) to 2 μl of chemical compound (3.2 mM) dissolved in 100% DMSO. This mixture was allowed to incubate at ambient temperature for 15 min and then reactions were initiated by addition of 15 μl of E. coli tRNA (20 μM), followed by a 2 hr incubation at room temperature (comparable to 1 hr at 37° C.). Reactions were stopped by the addition of 5 μl of 0.5 M EDTA. 200 μg of SPA beads (RNA Binding Beads (YSI), Perkin Elmer) in 150 μl of 300 mM citrate buffer (pH 6.2) were added. The plates were analyzed using a 1450 Microbeta (Jet) liquid scintillation and luminescence counter (Wallac). Assays to determine IC₅₀ values were as described above with the test compounds serially diluted from 200 μM to 0.4 μM. The concentration ranges of antibiotics as controls were as follows: spiramycin (0.02 μM to 20.0 μM), tylosin (0.02 μM to 20.0 μM), and fusidic acid (4 μM to 512 μM).

### PheRS Assays

SPA assays to determine inhibition of PheRS by chemical compounds were as described in Bullard et al. “Expression and characterization of a human mitochondrial phenylalanyl-tRNA synthetase” 1999, J. Mol. Biol. 288:567-577, which his incorporated herein by reference and in Ribble et al. Exceptions were that the enzyme mix was pre-incubated with 132 μM compound for 15 min prior to addition of tRNA. The reactions were stopped by the addition of 5 μl of 0.5 M EDTA. 400 μg of SPA beads (Ysi poly-L-lysine coated beads, Perkin Elmer) in 150 μl of 300 mM citrate buffer (pH 2.0) were added and the plates were analyzed as above.

To determine competition with ATP, IC₅₀s were determined in SPA reactions containing varying ATP concentrations. A mix (33 μl) containing 50 mM Tris-HCl (pH 7.5), 0.5 mM spermine, 8 mM MgCl2, 100 μM [³H]Phe (75 cpm/pmol), 0.05 mM DTT, indicated concentrations of ATP (25, 50, 100, 250, 500, 1000 μM), and 0.05 μM P. aeruginosa PheRS was added to the compound (2 μl). Final compound concentrations in the reactions ranged from 200 to 0.4 μM. The ATP concentrations ranged from approximately 8-fold below to 5-fold above the K_{M}. The mix was allowed to incubate at room temperature for 15 min. The reaction was started by addition of tRNA (15 μl, 80 μM). Positive controls contained only DMSO without compound. The reactions were for 30 minutes at 37° C. and stopped by the addition of 5 μl of 0.5 M EDTA.

To determine competition with phenylalanine the same assay was used. However, ATP was held at a constant concentrations of 2.0 mM in assays containing indicated concentrations of phenylalanine (25, 50, 100, 200, 300 μM). The Phe concentrations ranged from approximately 1-fold below to 10-fold above the K_{M}. Background amounts of free [³H]Phe in the absence of PheRS were insignificant.

### EF-Tu GDP Exchange Assay

Nitrocellulose binding assays were used to determine inhibition of GDP exchange by EF-Tu as previously described in Bullard et al. “Effects of domain exchanges between Escherichia coli and mammalian mitochondrial EF-Tu on interactions with guanine nucleotides, aminoacyl-tRNA and ribosomes” 1999, Biochim. Biophys. Acta 1446:102-114, which is incorporated herein by reference, with the exception that the enzyme (1 μM) was pre-incubated with 132 μM compound for 15 min prior to the addition of [³H]GDP. EF-Ts stimulates the exchange of GDP bound by EF-Tu. The ability of compounds to inhibit EF-Ts stimulation of GDP exchange by EF-Tu was measured in assays as described for EF-Tu/GDP exchange with the exceptions that EF-Ts was present at 0.05 μM and the time for the reaction was decreased from 30 min to 30 sec.

### EF-G GTPase Assay

Assays for ribosome-dependent GTP hydrolysis by EF-G were carried out in 50 μl reactions containing: 50 mM Tris-HCl (pH 7.5), 10 mM MgCl₂, 70 mM NH₄Cl, 1 mM dithiothreitol (DTT), 1.8 mM GTP, 0.4 μM P. aeruginosa ribosomes and 0.2 μM EF-G. Final concentrations of compounds in the assays were 132 μM. The amount of GTPase activity was determined by measurement of the amount of Pᵢ liberated using a colorimetric GTPase assay kit (Novus Biologicals) per manufacturer's directions.

### Eukaryotic Protein Synthesis Assay

Reactions to determine inhibitory effect of compounds on eukaryotic protein synthesis were carried out using wheat germ cell extracts as described in Ribble et al. Assays (50 μl) to test the inhibitory effect of compounds on the activity of human mitochondrial PheRS (hmPheRS) were carried out. The assay mix contained 50 mM Tris-HCl (pH 7.5), 1 mM spermine, 10 mM MgOAc, 2.5 mM ATP, 1 mM DTT, 75 μM Phe, and 0.5 μM hmPheRS. The mix was incubated for 15 min with various concentrations of compound, then the reaction was initiated by addition of tRNA^{Phe} (2 μM). The reaction was stopped by diluting into 3 ml of ice-cold 5% TCA followed by heating at 90° C. for 15 minutes and filtering through glass fiber filters as described. The concentrations of the compounds in these assays ranged from 0.8 to 200 μM and the concentration of the cycloheximide in the control reactions was from 0.3 to 300 μM.

### Microbiological Assays

Broth microdilution MIC testing was performed in 96-well microtiter plates according to Clinical Laboratory Standards Institute guideline M7-A7. MIC values were determined for E. coli (ATCC 25922), E. coli tolC mutant, Enterococcus faecalis (ATCC 29212), Haemophilus influenzae (ATCC 49766), Moraxella catarrhalis (ATCC 25238), Pseudomonas aeruginosa (ATCC 47085), Pseudomonas aeruginosa hypersensitive strain (ATCC 35151), Staphylococcus aureus (ATCC 29213), and Streptococcus pneumonia (ATCC 49619) from the American Type Culture Collection (Manassas, Va.).

Time-kill experiments were performed according to the CLSI guidelines M26-A. H. influenzae (ATCC 49766), E. faecalis ATCC 29212, and Staphylococcus aureus (ATCC 29213) were from the American Type Culture Collection (Manassas, Va.). Growth media were Brain Heart Infusion and Trypticase Soy Broth from Remel (Lenexa, Kans.). For the experiments, 10 mL of broth medium was inoculated with 0.1 mL of a fresh overnight culture and grown at 35° C. with shaking (200 rpm) for 2-3 hours. Pre-warmed flasks containing 10 mL of medium alone or 10 mL of medium containing a test compound at 4×MIC were then inoculated with 0.1 mL of the exponentially growing cultures. Samples were removed at 0, 2, 4, 6, and 24 h, and serial dilutions were plated on blood agar to allow for colony enumeration and calculation of the live cell density.

Using the above-described assays, a number of compounds were found that exhibit antibiotic activity. Specifically, six classes of compounds were found that are effective as antibiotics. The molecular target of the inhibitory compound was determined using the assays described above, if none of the accessory proteins were found to be inhibited by a compound, the ribosome was determined to be the target by default.

In one embodiment, a method of treating a bacterial infection in a subject comprises administering to the subject who would benefit from such treatment a therapeutically effective amount of a pharmaceutically acceptable formulation comprising an antibiotic compound as described below.

Class 1 Antibiotic Compounds—2′,4′-dihydroxychalcone Derivatives

(2E)-1-(2,4-Dihydroxyphenyl)-3-phenyl-2-propen-1-one was tested in the above-described assays and found to inhibit Phenylalanyl-tRNA synthetase (PheRS).

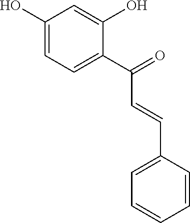 [SMILES: O=C(/C=C/c1ccccc1)c1ccc(O)cc1O]

### (2E)-1-(2,4-Dihydroxyphenyl)-3-phenyl-2-propen-1-one

Class 1 antibiotic compounds include (2E)-1-(2,4-Dihydroxyphenyl)-3-phenyl-2-propen-1-one and derivatives and analogues of this compound.

In one embodiment, antibiotic (2E)-1-(2,4-Dihydroxyphenyl)-3-phenyl-2-propen-1-one derivatives have the structure (I):

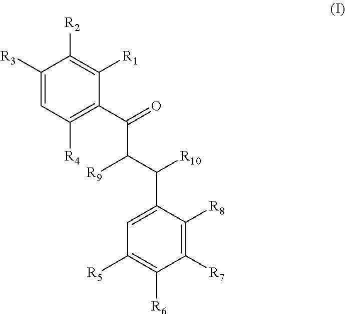

### Where:

R₁ is —H, —OH, —OR₁₁, —R₁₁, —CN, —CH₂-Ph, or -4-hydroxybenzyl;
R₂ is —H, —OH, —OR₁₁, —R₁₁, —CN, —CH₂-Ph, or -4-hydroxybenzyl;
R₃ is —H, —OH, —OR₁₁, —R₁₁, —CN, —CH₂-Ph, or -4-hydroxybenzyl;
R₄ is —H, —OH, —OR₁₁, —R₁, —CN, —CH₂-Ph, or -4-hydroxybenzyl;
R₅ is —H, —OH, —OR₁₁, —R₁₁, —CN, or —CH₂—CH═C(Me)₂;
R₆ is —H, —OH, —OR₁₁, —R₁₁, —CN, or —CH₂—CH═C(Me)₂;
R₇ is —H, —OH, —OR₁₁, —R₁₁, —CN, or —CH₂—CH═C(Me)₂;
R₈ is —H, —OH, —OR₁₁, —R₁₁, —CN, or —CH₂—CH═C(Me)₂;
R₉ and R₁₀ are —H or together form an additional bond;
R₁₁ is C₁-C₆ alkyl; and wherein at least one of R₁, R₂, R₃, and R₄ is not hydrogen.
In a specific embodiment, an antibiotic compound (I) has the structure (Ia):

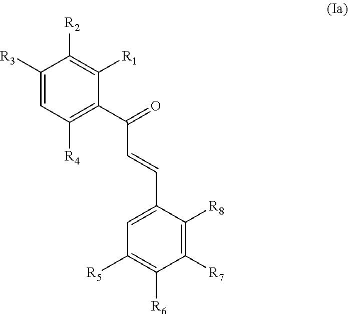

### Wherein:

R₁ is —H, —OH, —R₁₁, —CN, —CH₂-Ph, or -4-hydroxybenzyl;
R₂ is —H, —OH, —R₁₁, —CN, —CH₂-Ph, or -4-hydroxybenzyl;
R₃ is —H, —OH, —R₁₁, —CN, —CH₂-Ph, or -4-hydroxybenzyl;
R₄ is —H, —OH, —R₁, —CN, —CH₂-Ph, or -4-hydroxybenzyl;
R₅ is —H, —OH, —OR₁₁, —R₁₁, —CN, or —CH₂—CH═C(Me)₂;
R₆ is —H, —OH, —OR₁₁, —R₁₁, —CN, or —CH₂—CH═C(Me)₂;
R₇ is —H, —OH, —OR₁₁, —R₁₁, —CN, or —CH₂—CH═C(Me)₂;
R₈ is —H, —OH, —OR₁₁, —R₁₁, —CN, or —CH₂—CH═C(Me)₂;
R₁₁ is C₁-C₆ alkyl; and wherein at least one of R₁, R₂, R₃, and R₄ is not hydrogen. In another embodiment, the antibiotic compound has the structure of formula (Ia), wherein:
R₁ is —OH or —OR₁₁;
R₂ is —H, —OH, —R₁₁, or —CN;
R₃ is —H or —OR₁₁;

### R₄ is —H, or —OH;

R₅ is —H, —OH, or —OR₁₁;
R₆ is —H, —OH, —R₁₁, or —CN;
R₇ is —H, —OH, or —R₁₁;
R₈ is —H, —OH, or —OR₁₁; and
R₁₁ is C₁-C₆ alkyl.
Specific examples of antibiotic compounds having the structure of formula (I) include:

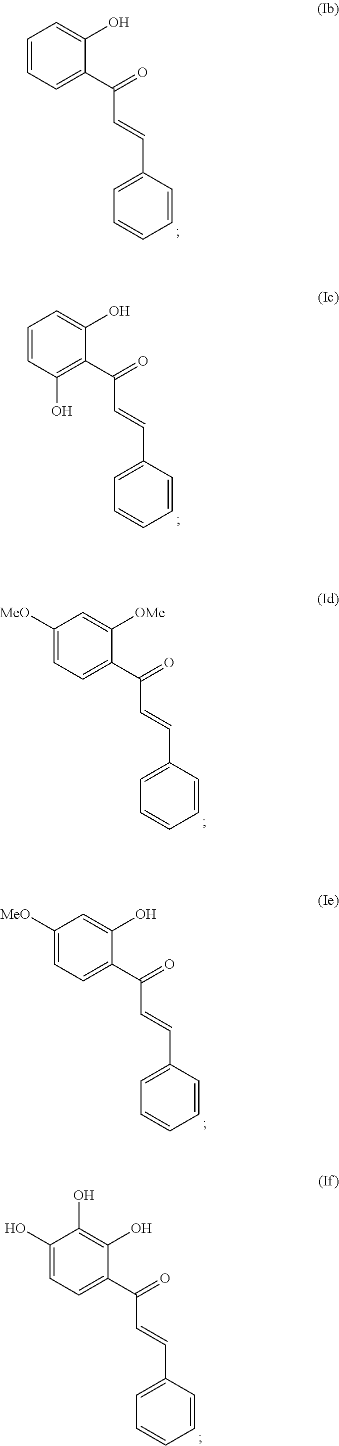 [SMILES: O=C(/C=C/c1ccccc1)c1ccccc1O ; O=C(/C=C/c1ccccc1)c1c(O)cccc1O ; COc1ccc(C(=O)/C=C/c2ccccc2)c(OC)c1 ; COc1ccc(C(=O)/C=C/c2ccccc2)c(O)c1 ; O=C(/C=C/c1ccccc1)c1ccc(O)c(O)c1O]

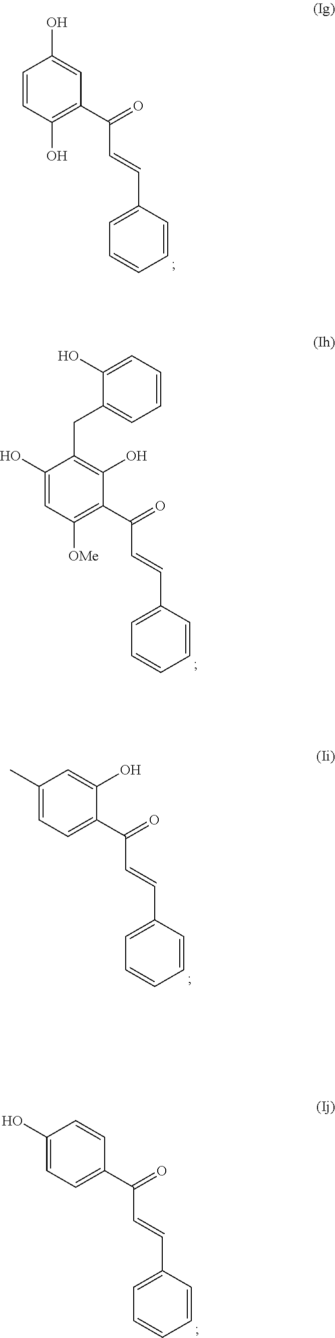 [SMILES: O=C(/C=C/c1ccccc1)c1cc(O)ccc1O ; COc1cc(O)c(Cc2ccccc2O)c(O)c1C(=O)/C=C/c1ccccc1 ; Cc1ccc(C(=O)/C=C/c2ccccc2)c(O)c1 ; O=C(/C=C/c1ccccc1)c1ccc(O)cc1]

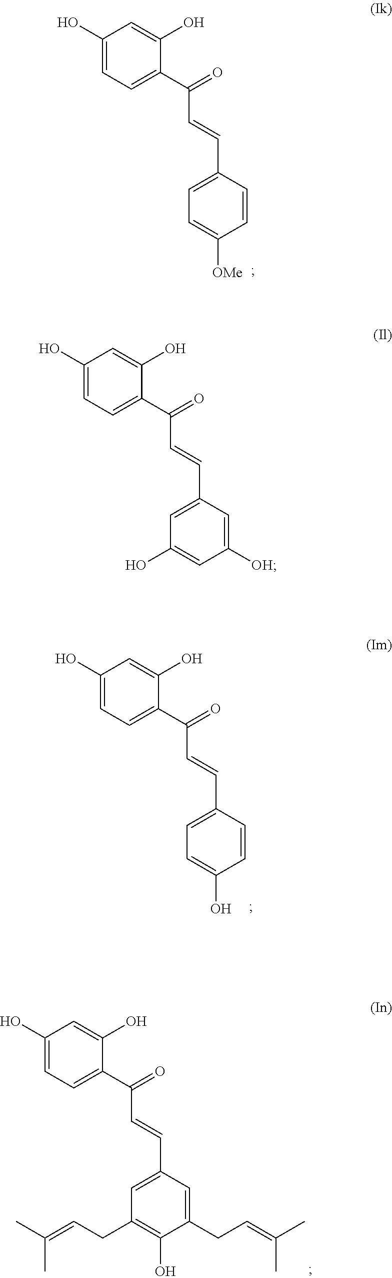 [SMILES: COc1ccc(/C=C/C(=O)c2ccc(O)cc2O)cc1 ; O=C(/C=C/c1cc(O)cc(O)c1)c1ccc(O)cc1O ; O=C(/C=C/c1ccc(O)cc1)c1ccc(O)cc1O ; CC(C)=CCc1cc(/C=C/C(=O)c2ccc(O)cc2O)cc(CC=C(C)C)c1O]

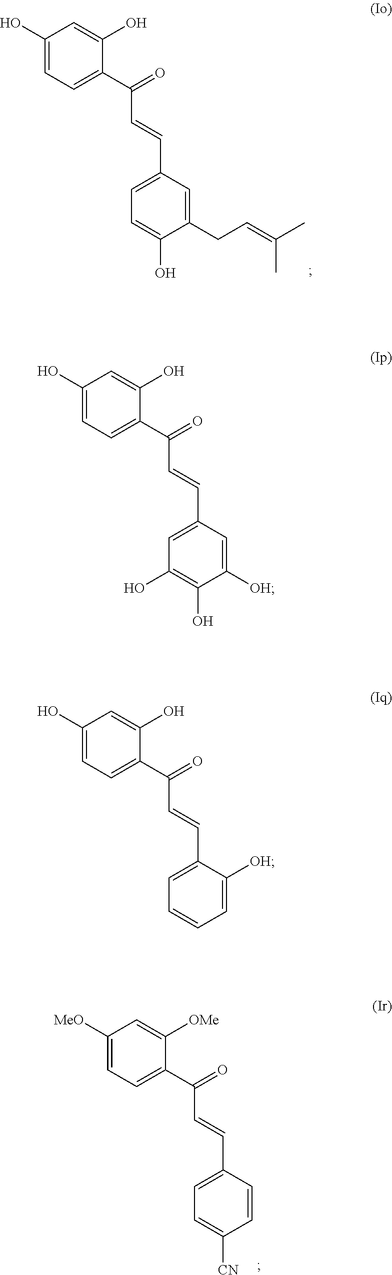 [SMILES: CC(C)=CCc1cc(/C=C/C(=O)c2ccc(O)cc2O)ccc1O ; O=C(/C=C/c1cc(O)c(O)c(O)c1)c1ccc(O)cc1O ; O=C(/C=C/c1ccccc1O)c1ccc(O)cc1O ; COc1ccc(C(=O)/C=C/c2ccc(C#N)cc2)c(OC)c1]

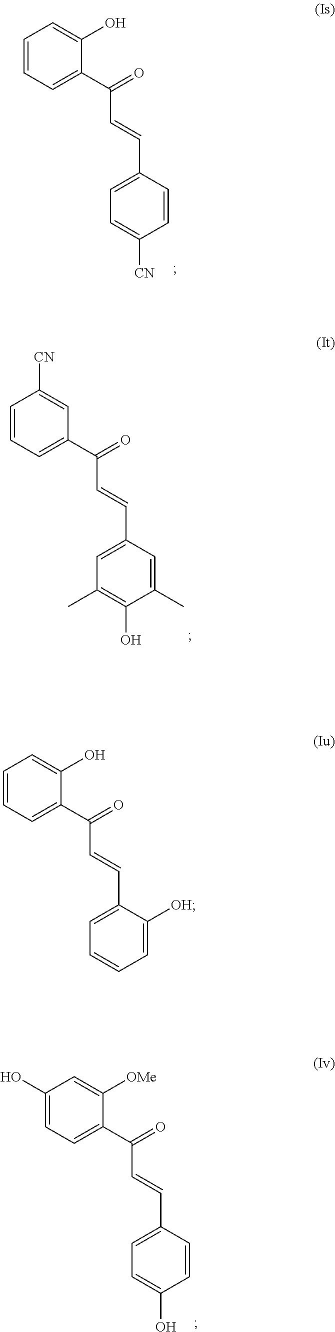 [SMILES: N#Cc1ccc(/C=C/C(=O)c2ccccc2O)cc1 ; Cc1cc(/C=C/C(=O)c2cccc(C#N)c2)cc(C)c1O ; O=C(/C=C/c1ccccc1O)c1ccccc1O ; COc1cc(O)ccc1C(=O)/C=C/c1ccc(O)cc1]

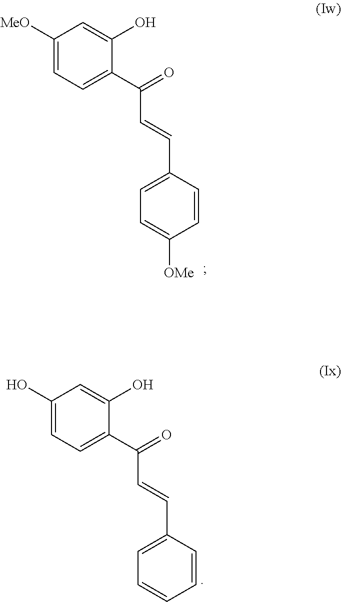 [SMILES: COc1ccc(/C=C/C(=O)c2ccc(OC)cc2O)cc1 ; O=C(/C=C/c1ccccc1)c1ccc(O)cc1O]

Analogues of (2E)-1-(2,4-Dihydroxyphenyl)-3-phenyl-2-propen-1-one that exhibit antibiotic activity include antibiotic compounds having the structure (II):

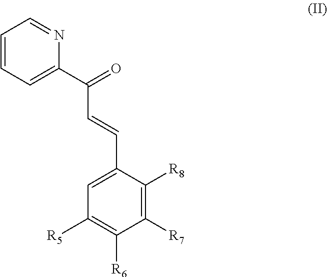

### Wherein:

R₅ is —H, —OH, —OR₁₁, —R₁₁, —CN, or —CH₂—CH═C(Me)₂;
R₆ is —H, —OH, —OR₁₁, —R₁₁, —CN, or —CH₂—CH═C(Me)₂;
R₇ is —H, —OH, —OR₁₁, —R₁₁, —CN, or —CH₂—CH═C(Me)₂;
R₈ is —H, —OH, —OR₁₁, —R₁₁, —CN, or —CH₂—CH═C(Me)₂;
R₁₁ is C₁-C₆ alkyl; and wherein at least one of R₁, R₂, R₃, and R₄ is not hydrogen.
In one embodiment, an antibiotic compound has the structure (II) where:
R₅ is —H, —OH, or —OR₁₁;
R₆ is —H, —OH, —R₁₁, or —CN;
R₇ is —H, —OH, or —R₁₁;
R₈ is —H, —OH, or —OR₁₁; and
R₁₁ is C₁-C₆ alkyl.
A specific antibiotic compound having the structure (II) is the compound:

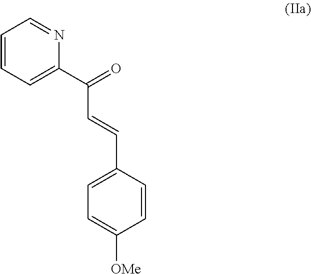 [SMILES: COc1ccc(/C=C/C(=O)c2ccccn2)cc1]

Analogues of (2E)-1-(2,4-Dihydroxyphenyl)-3-phenyl-2-propen-1-one that exhibit antibiotic activity include antibiotic compounds having the structure (III):

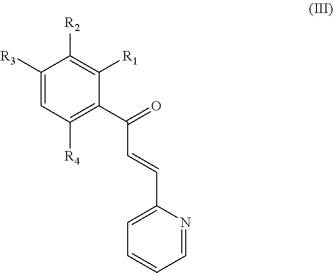

### Wherein:

R₁ is —H, —OH, —OR₁₁, —R₁₁, —CN, —CH₂-Ph, or -4-hydroxybenzyl;
R₂ is —H, —OH, —OR₁₁, —R₁₁, —CN, —CH₂-Ph, or -4-hydroxybenzyl;
R₃ is —H, —OH, —OR₁₁, —R₁₁, —CN, —CH₂-Ph, or -4-hydroxybenzyl;
R₄ is —H, —OH, —OR₁₁, —R₁, —CN, —CH₂-Ph, or -4-hydroxybenzyl;
R₁₁ is C₁-C₆ alkyl; and wherein at least one of R₁, R₂, R₃, and R₄ is not hydrogen.
In one embodiment, an antibiotic compound has the structure (III) wherein:
R₁ is —OH or —OR₁₁;
R₂ is —H, —OH, —OR₁₁, —R₁₁, or —CN;
R₃ is —H or —OR₁₁;

### R₄ is —H, or —OH;

R₁₁ is C₁-C₆ alkyl;
wherein at least one of R₁, R₂, R₃, and R₄ is not hydrogen.
A specific antibiotic compound having the structure (III) is the compound (IIIa):

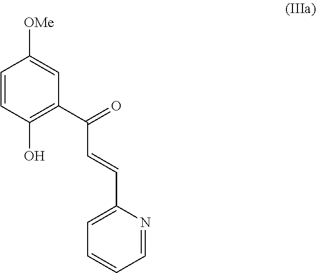 [SMILES: COc1ccc(O)c(C(=O)/C=C/c2ccccn2)c1]

Analogues of (2E)-1-(2,4-Dihydroxyphenyl)-3-phenyl-2-propen-1-one that exhibit antibiotic activity include antibiotic compounds having the structure (IV):

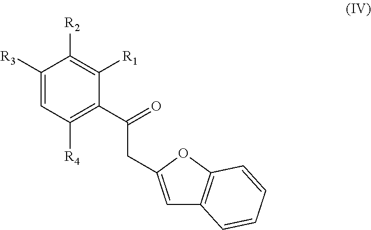

### Wherein:

R₁ is —H, —OH, —OR₁₁, —R₁₁, —CN, —CH₂-Ph, or -4-hydroxybenzyl;
R₂ is —H, —OH, —OR₁₁, —R₁₁, —CN, —CH₂-Ph, or -4-hydroxybenzyl;
R₃ is —H, —OH, —OR₁₁, —R₁₁, —CN, —CH₂-Ph, or -4-hydroxybenzyl;
R₄ is —H, —OH, —OR₁₁, —R₁, —CN, —CH₂-Ph, or -4-hydroxybenzyl;
R₁₁ is C₁-C₆ alkyl; and wherein at least one of R₁, R₂, R₃, and R₄ is not hydrogen.
In one embodiment, an antibiotic compound has the structure (IV) where:
R₁ is —OH or —OR₁₁;
R₂ is —H, —OH, —OR₁₁, —R₁₁, or —CN;
R₃ is —H or —OR₁₁;

### R₄ is —H, or —OH;

R₁₁ is C₁-C₆ alkyl; and wherein at least one of R₁, R₂, R₃, and R₄ is not hydrogen.
A specific antibiotic compound having the structure (IV) is the compound:

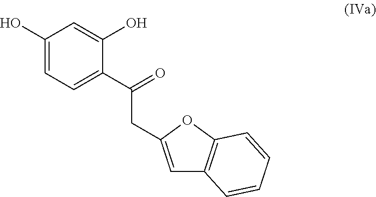 [SMILES: O=C(Cc1cc2ccccc2o1)c1ccc(O)cc1O]

Analogues of (2E)-1-(2,4-Dihydroxyphenyl)-3-phenyl-2-propen-1-one that exhibit antibiotic activity include antibiotic compounds having the structure (V):

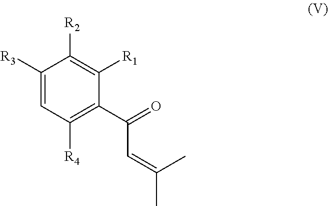

### Where:

R₁ is —H, —OH, —OR₁₁, —R₁₁, —CN, —CH₂-Ph, or -4-hydroxybenzyl;
R₂ is —H, —OH, —OR₁₁, —R₁₁, —CN, —CH₂-Ph, or -4-hydroxybenzyl;
R₃ is —H, —OH, —OR₁₁, —R₁₁, —CN, —CH₂-Ph, or -4-hydroxybenzyl;
R₄ is —H, —OH, —OR₁₁, —R₁, —CN, —CH₂-Ph, or -4-hydroxybenzyl;
R₁₁ is C₁-C₆ alkyl; and wherein at least one of R₁, R₂, R₃, and R₄ is not hydrogen.
In one embodiment, an antibiotic compound has the structure (V) where:
R₁ is —OH or —OR₁₁;
R₂ is —H, —OH, —OR₁₁, —R₁₁, or —CN;
R₃ is —H or —OR₁₁;

### R₄ is —H, or —OH;

R₁₁ is C₁-C₆ alkyl; and wherein at least one of R₁, R₂, R₃, and R₄ is not hydrogen.
A specific antibiotic compound having the structure (V) is the compound:

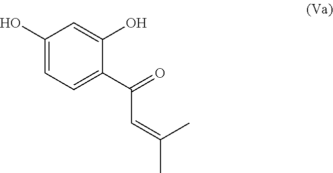

### Class 2 Antibiotic Compounds—Leoidin Derivatives

Methyl 2,4-dichloro-3,8-dihydroxy-1,6,9-trimethyl-11-oxo-11H-dibenzo[b,e][1,4]dioxepine-7-carboxylate (“leoidin”) was tested in the above-described assays and found to inhibit Phenylalanyl-tRNA synthetase (PheRS).

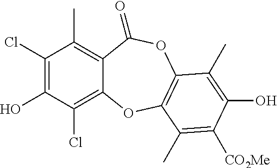 [SMILES: COC(=O)c1c(C)c2c(c(C)c1O)OC(=O)c1c(C)c(Cl)c(O)c(Cl)c1O2]

### Methyl 2,4-dichloro-3,8-dihydroxy-1,6,9-trimethyl-11-oxo-11H-dibenzo[b,e][1,4]dioxepine-7-carboxylate (“leoidin”)

Class 2 antibiotic compounds include leoidin and derivatives and analogues of this compound.

In one embodiment, antibiotic leoidin derivatives have the structure (VI):

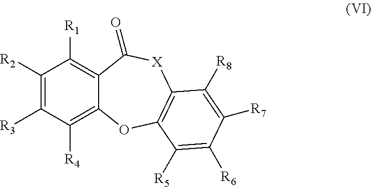

### Wherein:

X is O, NH, or NR₉
R₁ is —H; —R₉; —Cl; —F; —OH; —OR₉; —NH₂; —NHR₉; —N(R₉)₂; —CH₂—C(O)—CH₂—CH₂—CH₂—CH₂—CH₃; —CH₂—C(O)—CH₂—CH₂—CH₂—CH₃; -(E)-C(CH₃)═CH(CH₃); —(Z)—C(CH₃)═CH(CH₃); —CH₂—OH; —CHO; —NH—C(O)—NH—R₁₀; or —NH—C(O)—R₁₀;
R₂ is —H; —R₉; —Cl; —F; —OH; —OR₉; —NH₂; —NHR₉; —N(R₉)₂; —CH₂—C(O)—CH₂—CH₂—CH₂—CH₂—CH₃; —CH₂—C(O)—CH₂—CH₂—CH₂—CH₃; -(E)-C(CH₃)═CH(CH₃); —(Z)—C(CH₃)═CH(CH₃); —CH₂—OH; —CHO; —NH—C(O)—NH—R₁₀; or —NH—C(O)—R₁₀;
R₃ is —H; —R₉; —Cl; —F; —OH; —OR₉; —NH₂; —NHR₉; —N(R₉)₂; —CH₂—C(O)—CH₂—CH₂—CH₂—CH₂—CH₃; —CH₂—C(O)—CH₂—CH₂—CH₂—CH₃; -(E)-C(CH₃)═CH(CH₃); —(Z)—C(CH₃)═CH(CH₃); —CH₂—OH; —CHO; —NH—C(O)—NH—R₁₀; or —NH—C(O)—R₁₀;
R₄ is —H; —R₉; —Cl; —F; —OH; —OR₉; —NH₂; —NHR₉; —N(R₉)₂; —CH₂—C(O)—CH₂—CH₂—CH₂—CH₂—CH₃; —CH₂—C(O)—CH₂—CH₂—CH₂—CH₃; -(E)-C(CH₃)═CH(CH₃); —(Z)—C(CH₃)═CH(CH₃); —CH₂—OH; —CHO; —NH—C(O)—NH—R₁₀; or —NH—C(O)—R₁₀;
R₅ is —H; —R₉; —Cl; —F; —OH; —OR₉; —NH₂; —NHR₉; —N(R₉)₂; -(E)-C(CH₃)═CH(CH₃); (Z)—C(CH₃)═CH(CH₃); —CO₂H; —CO₂⁻; —CO₂R₉; —CH₂—OH; —CH₂—CH═C(CH₃)₂; —CHO; or —CH₂—C(O)—CH₃;
R₆ is —H; —R₉; —Cl; —F; —OH; —OR₉; —NH₂; —NHR₉; —N(R₉)₂; -(E)-C(CH₃)═CH(CH₃); (Z)—C(CH₃)═CH(CH₃); —CO₂H; —CO₂⁻; —CO₂R₉; —CH₂—OH; —CH₂—CH═C(CH₃)₂; —CHO; or —CH₂—C(O)—CH₃;
R₇ is —H; —R₉; —Cl; —F; —OH; —OR₉; —NH₂; —NHR₉; —N(R₉)₂; -(E)-C(CH₃)═CH(CH₃); (Z)—C(CH₃)═CH(CH₃); —CO₂H; —CO₂⁻; —CO₂R₉; —CH₂—OH; —CH₂—CH═C(CH₃)₂; —CHO; or —CH₂—C(O)—CH₃;
R₈ is —H; —R₉; —Cl; —F; —OH; —OR₉; —NH₂; —NHR₉; —N(R₉)₂; -(E)-C(CH₃)═CH(CH₃); (Z)—C(CH₃)═CH(CH₃); —CO₂H; —CO₂⁻; —CO₂R₉; —CH₂—OH; —CH₂—CH═C(CH₃)₂; —CHO; or —CH₂—C(O)—CH₃;
R₉ is C₁-C₆ alkyl;
R₁₀ is benzene or naphthalene substituted with one or more of the following: —H; —R₉; —Cl; —F; —OH; —OR₉; —NH₂; —NHR₉; —N(R₉)₂; and wherein at least one of R₁, R₂, R₃, and R₄ is not hydrogen.
   In a specific embodiment, the antibiotic compound has the structure of formula (VI), wherein:
X is O, NH, or NMe;
R₁ is —H; —R₉; or —CH₂—C(O)—CH₂—CH₂—CH₂—CH₂—CH₃
R₂ is —H; —Cl; —F; —OH; —OR₉; —NH₂; —NHR₉; —N(R₉)₂; or —CH₂—C(O)—CH₂—CH₂—CH₂—CH₃;
R₃ is —Cl; —F; —OH; or —OR₉;
R₄ is —H; —R₉; —Cl; —F; —OH; —OR₉; -(E)-C(CH₃)═CH(CH₃); —(Z)—C(CH₃)CH(CH₃); —CH₂—OH; or —CHO;
R₅ is —H; —R₉; —Cl; —F; -(E)-C(CH₃)═CH(CH₃); —(Z)—C(CH₃)═CH(CH₃); —CO₂H; —CO₂⁻; or CO₂R₉;
R₆ is —H; —Cl; —F; —OH; —OR₉; or —CO₂H; —CO₂⁻;
R₇ is —Cl; —F; —OH; —OR₉; or —CH₂—CH═C(CH₃)₂;
R₈ is —H; —R₉; —CH₂—OH; or CH₂—C(O)—CH₃; and
R₉ is C₁-C₆ alkyl.
   Specific examples of antibiotic compounds having the structure of formula (VI) include:

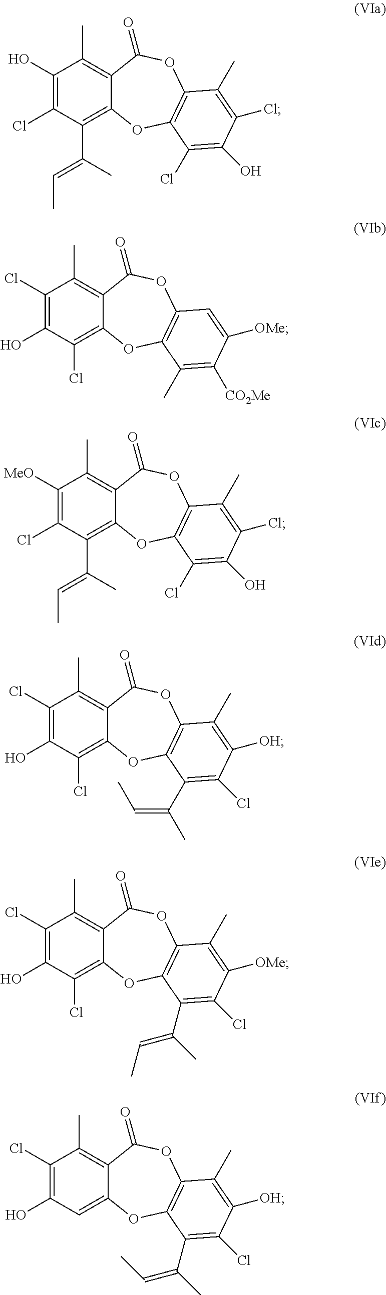

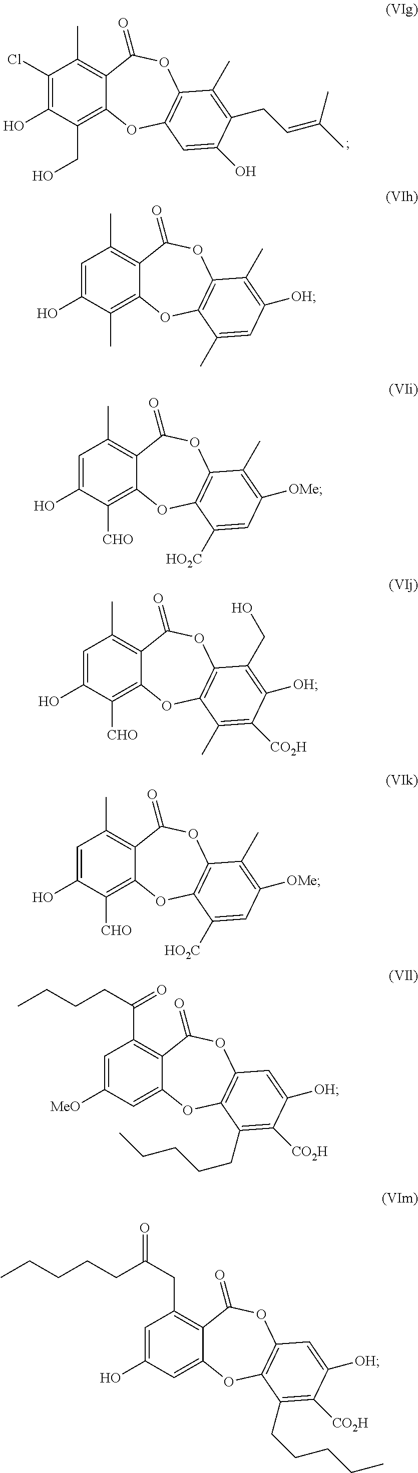

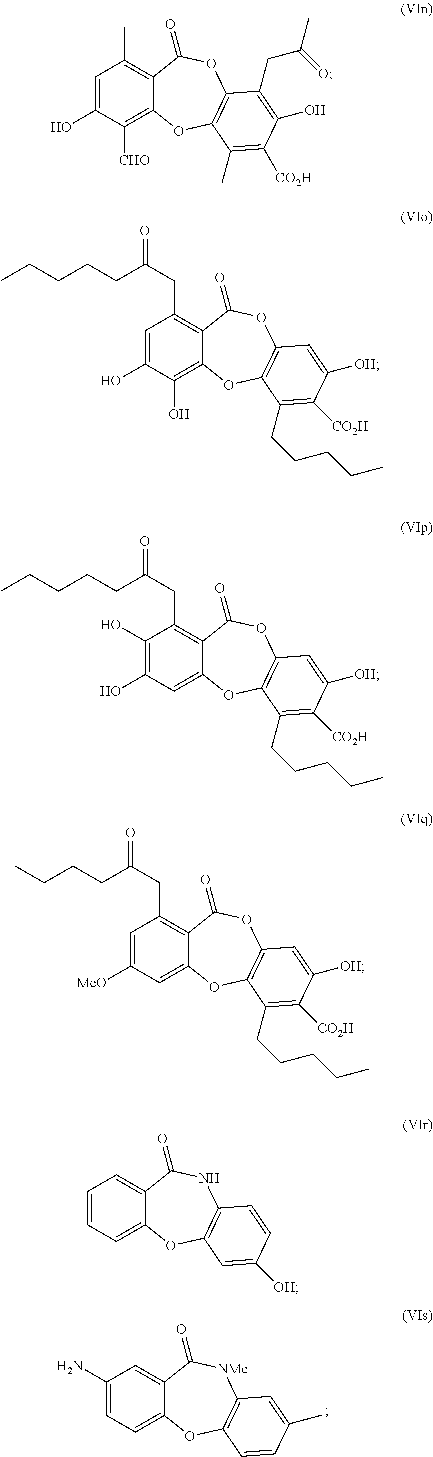

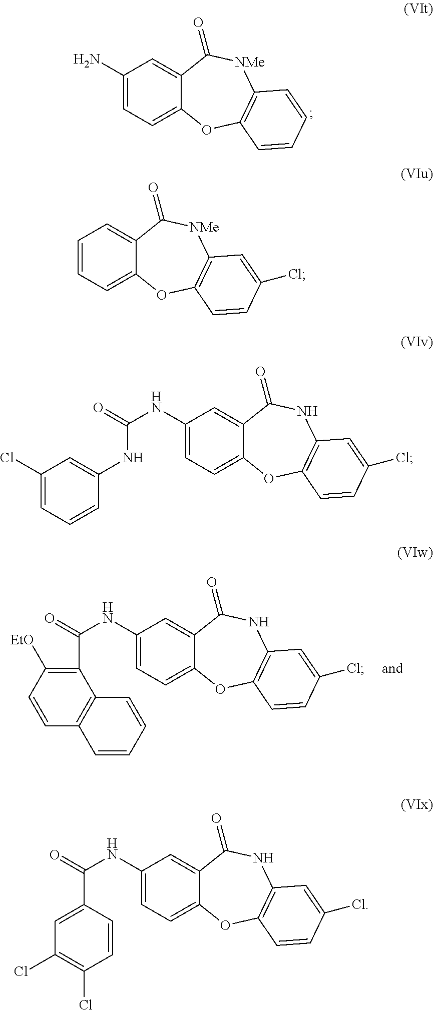 [SMILES: CN1C(=O)c2cc(N)ccc2Oc2ccccc21 ; CN1C(=O)c2ccccc2Oc2ccc(Cl)cc21 ; O=C(Nc1cccc(Cl)c1)Nc1ccc2c(c1)C(=O)Nc1cc(Cl)ccc1O2 ; CCOc1ccc2ccccc2c1C(=O)Nc1ccc2c(c1)C(=O)Nc1cc(Cl)ccc1O2 ; "and" ; O=C(Nc1ccc2c(c1)C(=O)Nc1cc(Cl)ccc1O2)c1ccc(Cl)c(Cl)c1]

Analogues of leoidin that exhibit antibiotic activity include antibiotic compounds having the structure (VII):

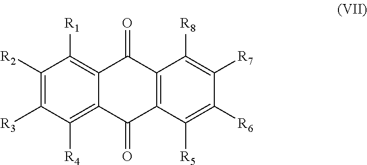

where each of R₁-R₈ is independently: —H; —R₉; —Cl; —F; —OH; —OR₉; —NH₂; —NHR₉; —N(R₉)₂; —CH₂—OH; or —CHO; and wherein at least one of R₁, R₂, R₃, and R₄ is not hydrogen.
Specific examples of antibiotic compounds having the structure of formula (VII) include:

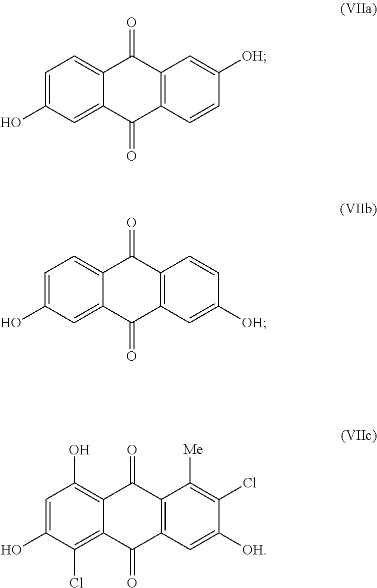 [SMILES: O=C1c2ccc(O)cc2C(=O)c2ccc(O)cc21 ; O=C1c2ccc(O)cc2C(=O)c2cc(O)ccc21 ; Cc1c(Cl)c(O)cc2c1C(=O)c1c(O)cc(O)c(Cl)c1C2=O]

Class 3 Antibiotic Compounds—{[4-chloro-3-(trifluoromethyl)phenyl]amino}-N-(3-prop-2-ynylthio(1,2,4-thiadiazol-5-yl))carboxamide Derivatives

{[4-chloro-3-(trifluoromethyl)phenyl]amino}-N-(3-prop-2-ynylthio(1,2,4-thiadiazol-5-yl))carboxamide was tested in the above-described assays and found to inhibit Phenylalanyl-tRNA synthetase (PheRS).

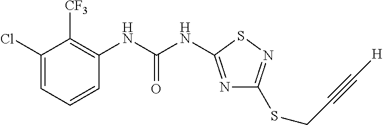 [SMILES: C#CCSc1nsc(NC(=O)Nc2cccc(Cl)c2C(F)(F)F)n1]

### {[4-chloro-3-(trifluoromethyl)phenyl]amino}-N-(3-prop-2-ynylthio(1,2,4-thiadiazol-5-yl))carboxamide

Class 3 antibiotic compounds include {[4-chloro-3-(trifluoromethyl)phenyl]amino}-N-(3-prop-2-ynylthio(1,2,4-thiadiazol-5-yl))carboxamide and derivatives and analogues of this compound.

In one embodiment, antibiotic {[4-chloro-3-(trifluoromethyl)phenyl]amino}-N-(3-prop-2-ynylthio(1,2,4-thiadiazol-5-yl))carboxamide derivatives have the structure (VIII):

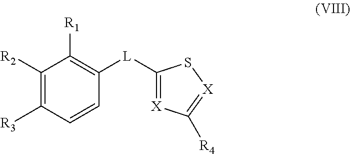

### Wherein:

L is: —NH—C(O)—NH—; —C(O)—NH—; —C(CH₃)—NH—C(O)—NH—; —CH₂—C(O)—NH—; —CH₂—CH(OH)—CH₂—NH—C(O)—CH₂—
X is: N; CH; CMe; C—CO₂H; C—CO₂R; C—CONH₂; C—CONHR₅; or C—CON(R₅)₂;
Y is: N; CH; CMe; C—CO₂H; or C—CO₂R; C—CONH₂; C—CONHR₅; or C—CON(R₅)₂;
R₁ is: —H; CF₃; —R₅; —Cl; —F; —OH; or —OR₅;
R₂ is: —H; CF₃; —R₅; —Cl; —F; —OH; or —OR₅;
R₃ is: —H; CF₃; —R₅; —Cl; —F; —OH; or —OR₅;
R₄ is: —R₅; —S—R₅; —S—CH₂—CH═CH₂; —S—CH₂—C≡CH; phenyl; or furanyl;
R₅ is C₁-C₆ alkyl; and wherein at least one of R₁, R₂, R₃, and R₄ is not hydrogen.
   In an embodiment, an antibiotic compound has the structure (VIIIa):

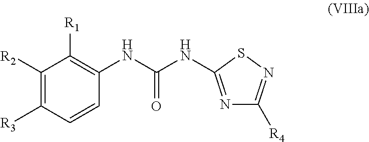

### Wherein:

R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅;
R₄ is: —S—R₅;
R₅ is C₁-C₆ alkyl; and wherein at least one of R₁, R₂, R₃, and R₄ is not hydrogen.
In an embodiment, an antibiotic compound has the structure (VIIIb):

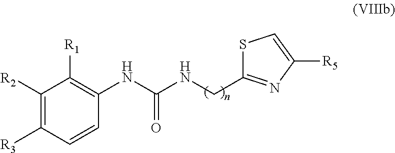

### Wherein:

R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅;
R₅ is C₁-C₆ alkyl; and wherein at least one of R₁, R₂, R₃, and R₄ is not hydrogen.
In an embodiment, an antibiotic compound has the structure (VIIIc):

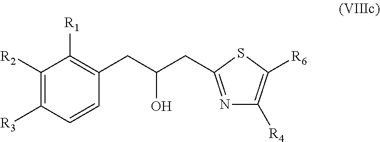

### Wherein:

R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅;
R₄ is: —R₅ or -Ph;
R₅ is C₁-C₆ alkyl;
R₆ is —H; —CO₂H; —CO₂R₅; and wherein at least one of R₁, R₂, R₃, and R₄ is not hydrogen.
In an embodiment, an antibiotic compound has the structure (VIIId):

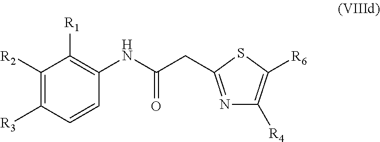

### Wherein:

R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅;
R₄ is: —R₅ or -Ph;
R₅ is C₁-C₆ alkyl;
R₆ is —H; —CO₂H; —CO₂R₅; and wherein at least one of R₁, R₂, R₃, and R₄ is not hydrogen.
In an embodiment, an antibiotic compound has the structure (VIIIe):

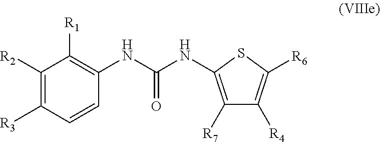

### Wherein:

R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅;
R₄ is: —R₅; -Ph; or furanyl;
R₅ is C₁-C₆ alkyl;
R₆ is —H; —CO₂H; —CO₂R₅; —CONH₂; —CONR₅; —CON(R₅)₂;
R₇ is —H; —CO₂H; —CO₂R₅; —CONH₂; —CONR₅; —CON(R₅)₂;
wherein at least one of R₁, R₂, R₃, and R₄ is not hydrogen.
Specific examples of antibiotic compounds having the structure of formula (VIII) include:

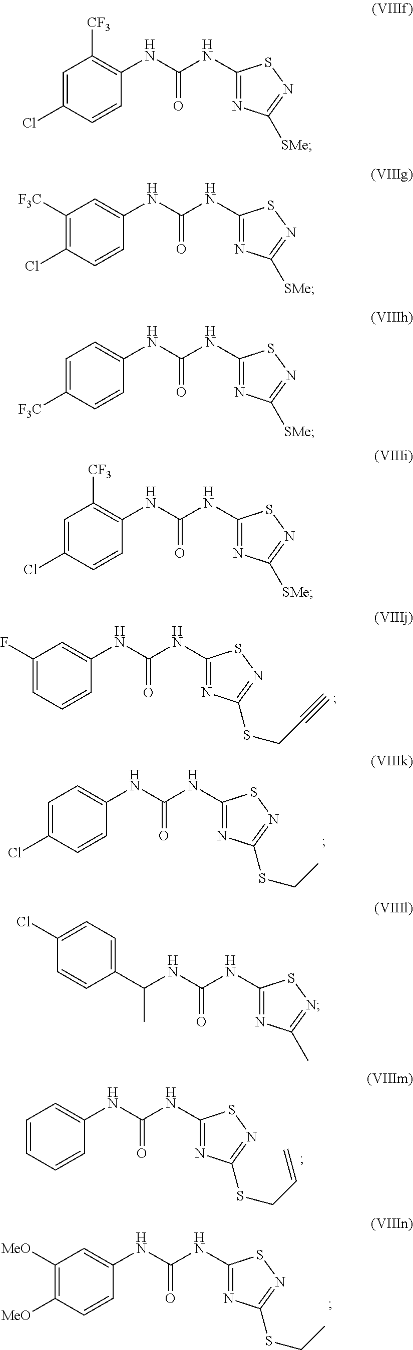

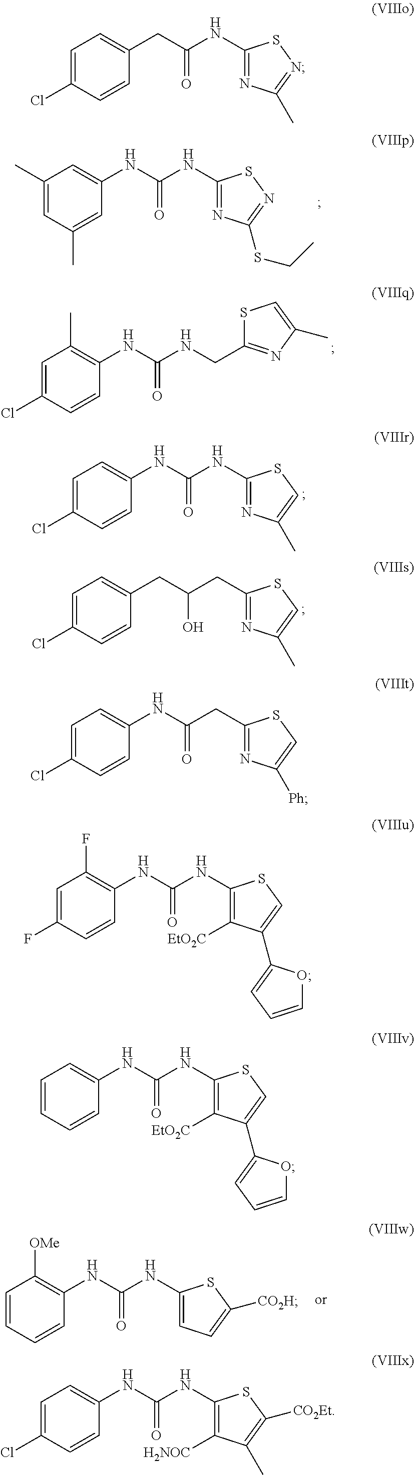

Analogues of {[4-chloro-3-(trifluoromethyl)phenyl]amino}-N-(3-prop-2-ynylthio(1,2,4-thiadiazol-5-yl))carboxamide that exhibit antibiotic activity include antibiotic compounds having the structure (IX):

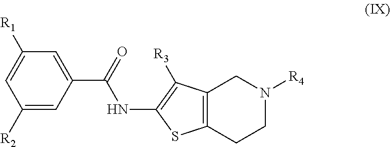

### Wherein:

R₁ is: —H; —CF₃; —R₅; —Cl; —F; —OH; or —OR₅;
R₂ is: —H; —CF₃; —R₅; —Cl; —F; —OH; or —OR₅;
R₃ is —H; —CO₂H; —CO₂R₅; —CONH₂; —CONR₅; or —CON(R₅)₂;
R₄ is —H or —R₅;
R₅ is C₁-C₆ alkyl; and wherein at least one of R₁, R₂, R₃, and R₄ is not hydrogen.
Specific examples of antibiotic compounds having the structure of formula (IX) include:

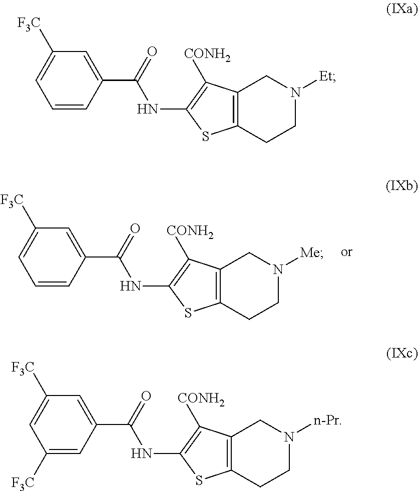

Class 4 Antibiotic Compounds—N-[(1E)-2-(5-nitro(2-furyl))-1-azavinyl](2-hydroxy-3,5-dinitrophenyl)carboxamide Derivatives

N-[(1E)-2-(5-nitro(2-furyl))-1-azavinyl](2-hydroxy-3,5-dinitrophenyl)carboxamide was tested in the above-described assays and found to inhibit Phenylalanyl-tRNA synthetase (PheRS).

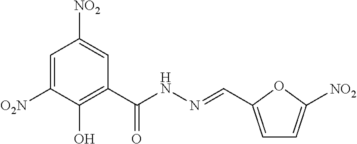 [SMILES: O=C(N/N=C/c1ccc([N+](=O)[O-])o1)c1cc([N+](=O)[O-])cc([N+](=O)[O-])c1O]

### N-[(1E)-2-(5-nitro(2-furyl))-1-azavinyl](2-hydroxy-3,5-dinitrophenyl)carboxamide

Class 4 antibiotic compounds include N-[(1E)-2-(5-nitro(2-furyl))-1-azavinyl](2-hydroxy-3,5-dinitrophenyl)carboxamide and derivatives and analogues of this compound.

In one embodiment, antibiotic N-[(1E)-2-(5-nitro(2-furyl))-1-azavinyl](2-hydroxy-3,5-dinitrophenyl)carboxamide derivatives have the structure (XI):

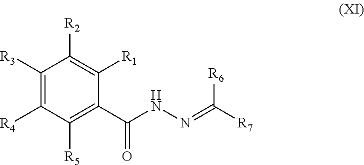

wherein:
R₁ is: —H; —NO₂; —F; —Cl; —Br; OH; —OR₈; or —CF₃
R₂ is: —H; —NO₂; —F; —Cl; —Br; OH; —OR₈; or —CF₃
R₃ is: —H; —NO₂; —F; —Cl; —Br; OH; —OR₈; or —CF₃ or R₂ and R₃ combine to form an aromatic ring;
R₄ is: —H; —NO₂; —F; —Cl; —Br; OH; —OR₈; or —CF₃;
R₅ is: —H; —NO₂; —F; —Cl; —Br; OH; —OR₈; or —CF₃;
wherein at least one of R₁, R₂, R₃, R₄, and R₅ is not hydrogen;
R₆ is: —H or C₁-C₆ alkyl;
R₇ is an aryl or heterocyclic substituent;
R₈ is C₁-C₆ alkyl; and
wherein when R₇ is aryl, R₇ has the structure:

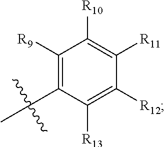

wherein when R₇ is a heterocyclic ring, exemplary heterocyclic rings include, but are not limited to:

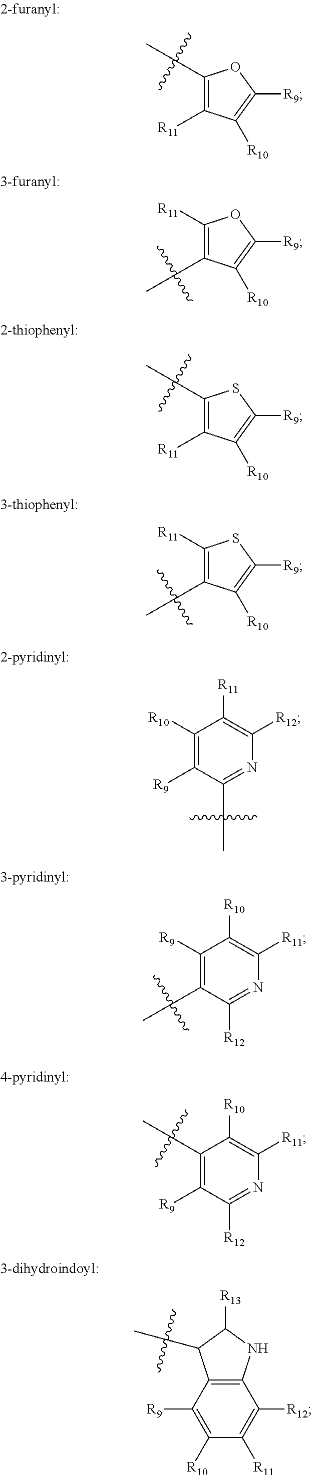

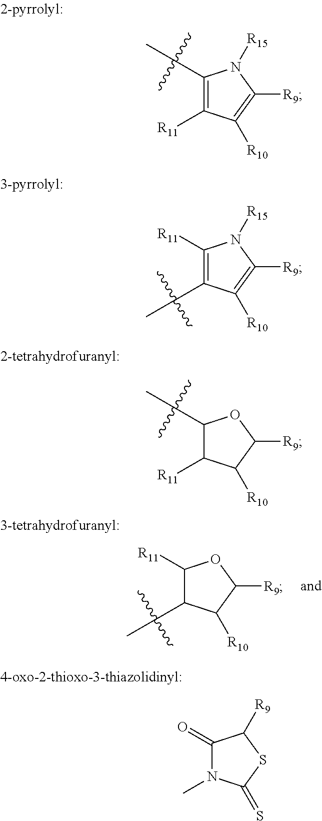

wherein each R₉, R₁₀, R₁₁, R₁₂, and R₁₃ are independently: —H; —NO₂; —F; —Cl; —Br; OH; —OR₈; or —CF₃;
and wherein R₁₅ is H, alkyl, or aryl (as defined above).
In a specific embodiment, an antibiotic compound has the structure (XI) wherein:

### R₁ is: —H;

R₂ is: —H; —NO₂; —Br;
R₃ is: —H; —OR₈;
or R₂ and R₃ combine to form an aromatic ring;
R₄ is: —H; —NO₂; —OR₈;
R₅ is: —H; —OH; —OR₈;
R₆ is: —H or C₁-C₆ alkyl;
R₇ is an aryl or heterocyclic substituent;
R₈ is C₁-C₆ alkyl;
wherein, when R₅ is —OH, R₄ is —H or —OR₈; and
wherein at least one of R₁, R₂, R₃, R₄, and R₅ is not hydrogen.
In a specific embodiment, an antibiotic compound has the structure:

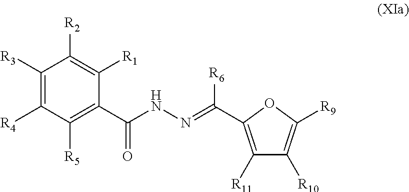

wherein:

### R₁ is: —H;

R₂ is: —H; —NO₂; —Br;
R₃ is: —H; —OR₈;
or R₂ and R₃ combine to form an aromatic ring;
R₄ is: —H; —NO₂; —OR₈;
R₅ is: —H; —OH; —OR₈;
R₆ is: —H or C₁-C₆ alkyl;
R₇ is an aryl or heterocyclic substituent;
R₈ is C₁-C₆ alkyl;
wherein, when R₅ is —OH, R₄ is —H or —OR₈;
wherein at least one of R₁, R₂, R₃, R₄, and R₅ is not hydrogen; and
wherein each R₉, R₁₀, and R₁₁ are independently: —H; —NO₂; —F; —Cl; —Br; OH; —OR₈; or —CF₃.
Specific examples of antibiotic compounds having the structure of formula (XI) include:

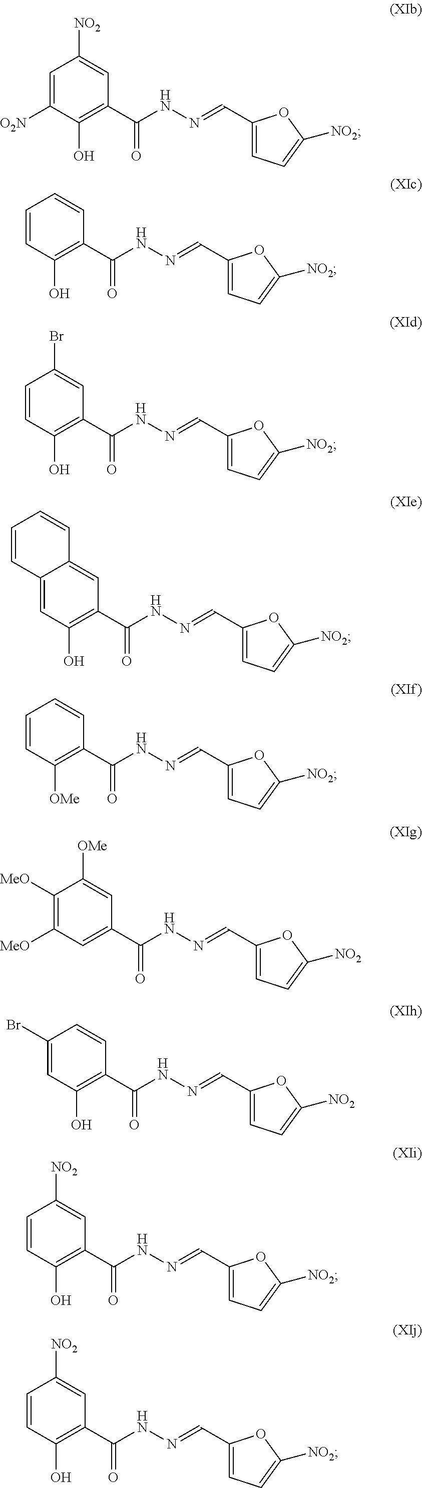

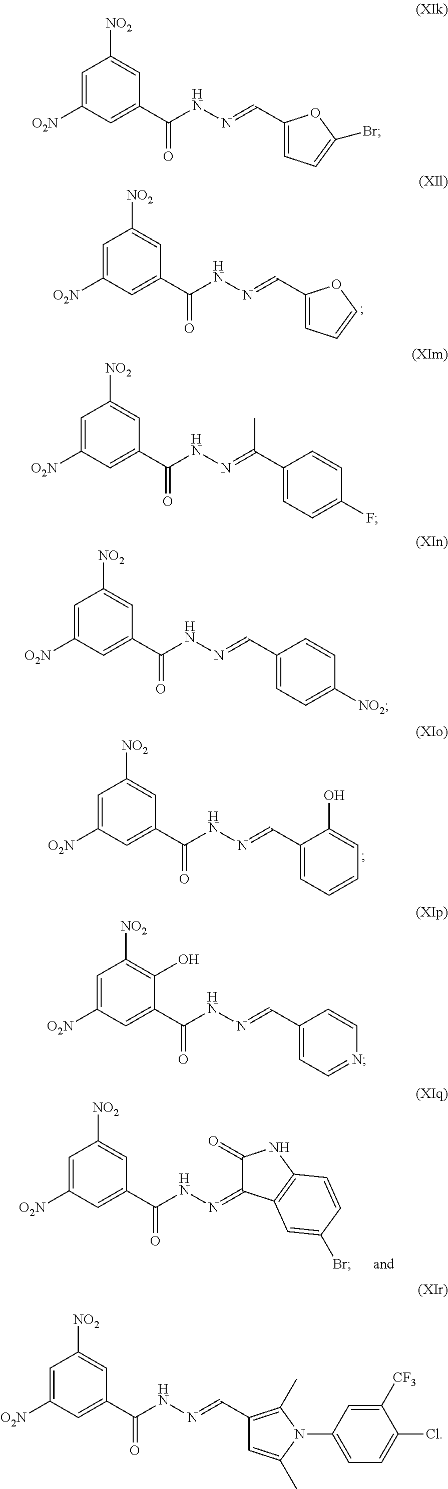

Analogues of N-[(1E)-2-(5-nitro(2-furyl))-1-azavinyl](2-hydroxy-3,5-dinitrophenyl)carboxamide that exhibit antibiotic activity include antibiotic compounds having the structure (XII):

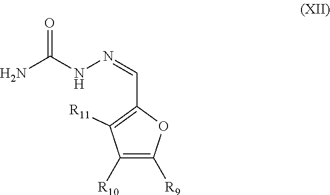

where each R₉, R₁₀, and R₁₁ are independently: —H; —NO₂; —NH₂; —NH(R₁₆); —N(R₁₆)₂; —F; —Cl; —Br; —OH; —OR₈; or —CF₃;
wherein at least one of R₉, R₁₀, and R₁₁ is not hydrogen; and
wherein R₁₆ is C₁-C₆ alkyl.
An exemplary compound of having the structure (XII) is the compound (XIIa)

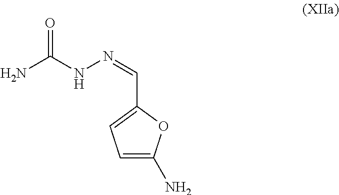 [SMILES: NC(=O)N/N=C\c1ccc(N)o1]

Analogues of N-[(1E)-2-(5-nitro(2-furyl))-1-azavinyl](2-hydroxy-3,5-dinitrophenyl)carboxamide that exhibit antibiotic activity include antibiotic compounds having the structure (XIII):

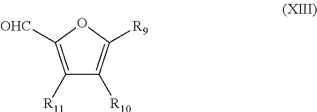

where each R₉, R₁₀, and R₁₁ are independently: —H; —NO₂; —NH₂; —NH(R₁₆); —N(R₁₆)₂; —F; —Cl; —Br; —OH; —OR₈; or —CF₃;
wherein at least one of R₉, R₁₀, and R₁₁ is not hydrogen; and
wherein R₁₆ is C₁-C₆ alkyl.
An exemplary compound of having the structure (XIII) is the compound (XIIIa)

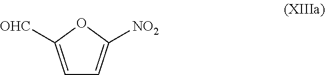

Analogues of N-[(1E)-2-(5-nitro(2-furyl))-1-azavinyl](2-hydroxy-3,5-dinitrophenyl)carboxamide that exhibit antibiotic activity include antibiotic compounds having the structure (XIV):

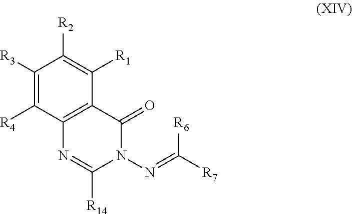

### Wherein:

R₁ is: —H; —NO₂; —F; —Cl; —Br; OH; —OR₈; or —CF₃
R₂ is: —H; —NO₂; —F; —Cl; —Br; OH; —OR₈; or —CF₃
R₃ is: —H; —NO₂; —F; —Cl; —Br; OH; —OR₈; or —CF₃
or R₂ and R₃ combine to form an aromatic ring;
R₄ is: —H; —NO₂; —F; —Cl; —Br; OH; —OR₈; or —CF₃;
wherein at least one of R₁, R₂, R₃, and R₄ is not hydrogen
R₆ is: —H or C₁-C₆ alkyl;
R₇ is an aryl or heterocyclic substituent; and
R₁₄ is C₁-C₆ alkyl.
An exemplary compound of having the structure (XIV) is the compound (XIVa)

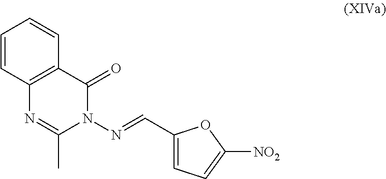 [SMILES: Cc1nc2ccccc2c(=O)n1/N=C/c1ccc([N+](=O)[O-])o1]

Analogues of N-[(1E)-2-(5-nitro(2-furyl))-1-azavinyl](2-hydroxy-3,5-dinitrophenyl)carboxamide that exhibit antibiotic activity include antibiotic compounds having the structure (XV):

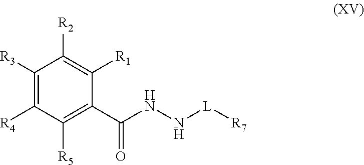

### Wherein:

R₁ is: —H; —NO₂; —F; —Cl; —Br; OH; —OR₈; or —CF₃
R₂ is: —H; —NO₂; —F; —Cl; —Br; OH; —OR₈; or —CF₃
R₃ is: —H; —NO₂; —F; —Cl; —Br; OH; —OR₈; or —CF₃
or R₂ and R₃ combine to form an aromatic ring;
R₄ is: —H; —NO₂; —F; —Cl; —Br; OH; —OR₈; or —CF₃;
R₅ is: —H; —NO₂; —F; —Cl; —Br; OH; —OR₈; or —CF₃;
wherein at least one of R₁, R₂, R₃, R₄, and R₅ is not hydrogen
R₇ is alkyl, alkenyl, aryl or a heterocyclic substituent;
R₈ is C₁-C₆ alkyl; and
where L is: —C(O)—NH—CH₂—; —C(O)—NH—CH₂—CH₂—; —C(O)—CH₂—O—; ═CH—CH═CH—; —C(O)—; CH—; or ═C(Me)-.
Exemplary compounds having the structure (XV) include:

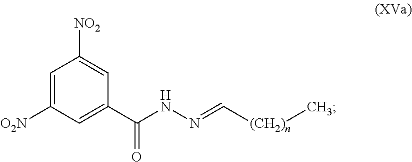

where n is 1-20

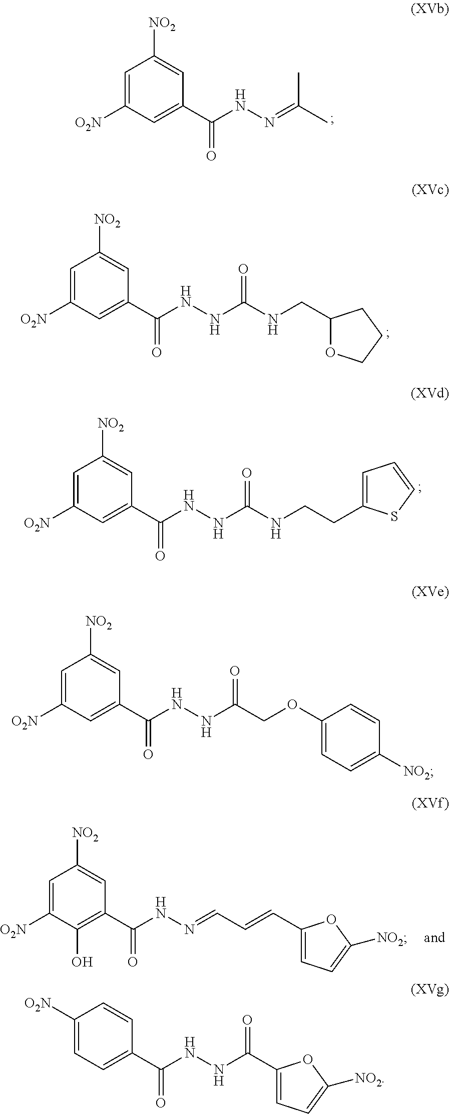

Analogues of N-[(1E)-2-(5-nitro(2-furyl))-1-azavinyl](2-hydroxy-3,5-dinitrophenyl)carboxamide that exhibit antibiotic activity include antibiotic compounds having the structure (XVI):

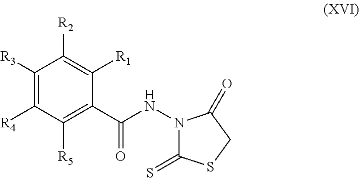

wherein:
R₁ is: —H; —NO₂; —F; —Cl; —Br; OH; —OR₈; or —CF₃
R₂ is: —H; —NO₂; —F; —Cl; —Br; OH; —OR₈; or —CF₃
R₃ is: —H; —NO₂; —F; —Cl; —Br; OH; —OR₈; or —CF₃
or R₂ and R₃ combine to form an aromatic ring;
R₄ is: —H; —NO₂; —F; —Cl; —Br; OH; —OR₈; or —CF₃;
R₅ is: —H; —NO₂; —F; —Cl; —Br; OH; —OR₈; or —CF₃;
wherein at least one of R₁, R₂, R₃, R₄, and R₅ is not hydrogen; and
R₈ is C₁-C₆ alkyl.
An exemplary compound having the structure (XVI) includes compound (XVIa):

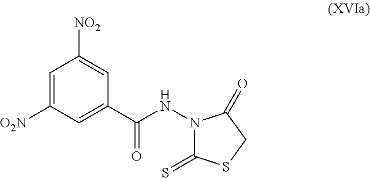 [SMILES: O=C(NN1C(=O)CSC1=S)c1cc([N+](=O)[O-])cc([N+](=O)[O-])c1]

Class 5 Antibiotic Compounds—2-[1,5-bis(5-nitrofuran-2-yl)penta-1,4-dien-3-ylideneamino]guanidine hydrochloride Derivatives

2-[1,5-bis(5-nitrofuran-2-yl)penta-1,4-dien-3-ylideneamino]guanidine hydrochloride was tested in the above-described assays and found to be a ribosomal inhibitor.

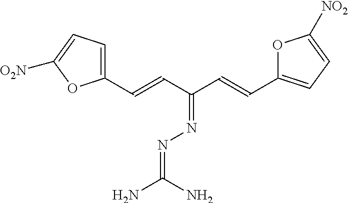 [SMILES: NC(N)=NN=C(/C=C/c1ccc([N+](=O)[O-])o1)/C=C/c1ccc([N+](=O)[O-])o1]

### 2-[1,5-bis(5-nitrofuran-2-yl)penta-1,4-dien-3-ylideneamino]guanidine hydrochloride

Class 5 antibiotic compounds include 2-[1,5-bis(5-nitrofuran-2-yl)penta-1,4-dien-3-ylideneamino]guanidine hydrochloride and derivatives and analogues of this compound.

In one embodiment, antibiotic 2-[1,5-bis(5-nitrofuran-2-yl)penta-1,4-dien-3-ylideneamino]guanidine hydrochloride derivatives have the structure (XVII):

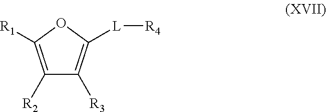

### Wherein:

R₁ is: —H; —NO₂; —F; —Cl; —Br; OH; —OR₈; or —CF₃
R₂ is: —H; —NO₂; —F; —Cl; —Br; OH; —OR₈; or —CF₃
R₃ is: —H; —NO₂; —F; —Cl; —Br; OH; —OR₈; or —CF₃
R₄ is aryl or a substituted or unsubstituted heterocyclic substituent;
R₈ is C₁-C₆ alkyl; and
wherein L is: -(E)-CH═CH—C(N—N═C(NH₂)₂)-(E)-CH═CH—;
-(E)-CH═CH—C(N—N═C(NH₂)₂)—(Z)—CH═CH—;
—(Z)—CH═CH—C(N—N═C(NH₂)₂)-(E)-CH═CH—;
—(Z)—CH═CH—C(N—N═C(NH₂)₂)—(X)—CH═CH—;
-(E)-CH═CH—C(O)-(E)-CH═CH—;
-(E)-CH═CH—C(O)—(Z)—CH═CH—;
—(Z)—CH═CH—C(O)-(E)-CH═CH—;
—(Z)—CH═CH—C(O)—(Z)—CH═CH—;
-(E)-CH═CH—CH═N—NH—C(S)—NH—;
—(Z)—CH═CH—CH═N—NH—C(S)—NH—;
-(E)-CH CH—;
—(Z)—CH═CH—;
-(E)-CH═CH—C(Me)=N—N═CH—;
—(Z)—CH═CH—C(Me)=N—N═CH—;

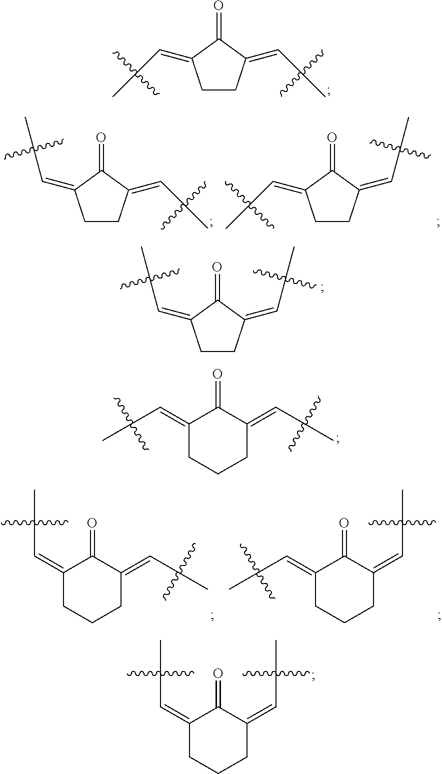

When R₄ is aryl, R₄ has the structure:

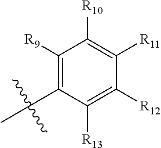

In some embodiments, R₄ is a substituted or unsubstituted furanyl, substituted or unsubstituted thiophenyl; substituted or unsubstituted pyrrolyl.

When R₄ is a furanyl, R₄ can be 2-furanyl or 3-furanyl having the structure:

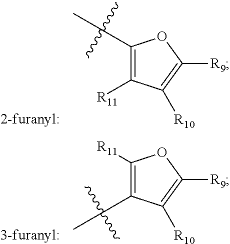

When R₄ is thiophenyl, R₄ can be 2-thiophenyl or 3-thiophenyl having the structure:

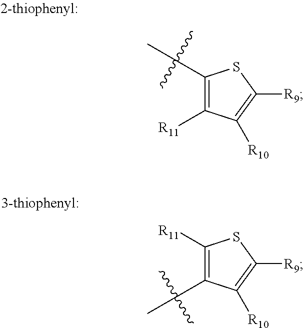

When R₄ is pyrrolyl, R₄ can be 2-pyrrolyl or 3-pyrrolyl having the structure:

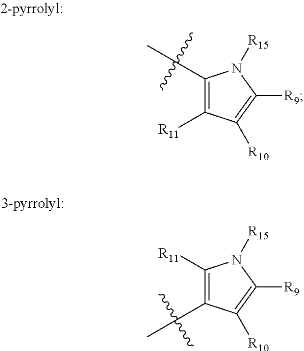

where each R₉, R₁₀, R₁₁, R₁₂, and R₁₃ are independently: —H; —NO₂; —F; —Cl; —Br; OH; —OR₈; —OBn; or —CF₃; and
where R₁₅ is H, alkyl, or aryl (as defined above).
In a specific embodiment, an antibiotic compound has the structure (XVIIa):

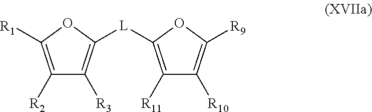

### Wherein:

R₁ is: —H; —NO₂; —F; —Cl; —Br; OH; —OR₈; or —CF₃
R₂ is: —H; —NO₂; —F; —Cl; —Br; OH; —OR₈; or —CF₃
R₃ is: —H; —NO₂; —F; —Cl; —Br; OH; —OR₈; or —CF₃
R₈ is C₁-C₆ alkyl;
R₉ is: —H; —NO₂; —F; —Cl; —Br; OH; —OR₈; or —CF₃
R₁₀ is: —H; —NO₂; —F; —Cl; —Br; OH; —OR₈; or —CF₃
R₁₁ is: —H; —NO₂; —F; —Cl; —Br; OH; —OR₈; or —CF₃; and
wherein L is: -(E)-CH═CH—C(N—N═C(NH₂)₂)-(E)-CH═CH—;
-(E)CH═CH—C(N—N═C(NH₂)₂)—(Z)—CH═CH—;
—(Z)—CH═CH—C(N—N═C(NH₂)₂)-(E)-CH═CH—;
—(Z)—CH═CH—C(N—N═C(NH₂)₂)—(X)—CH═CH—;
-(E)-CH═CH—C(O)-(E)-CH═CH—;
-(E)-CH═CH—C(O)—(Z)—CH═CH—;
—(Z)—CH═CH—C(O)-(E)-CH═CH—;
—(Z)—CH═CH—C(O)—(Z)—CH═CH—;
-(E)-CH═CH—CH═N—NH—C(S)—NH—;
—(Z)—CH═CH—CH═N—NH—C(S)—NH—;
-(E)-CH CH—;
—(Z)—CH═CH—;
-(E)-CH═CH—C(Me)N—N═CH—;
—(Z)—CH═CH—C(Me)=N—N═CH—;

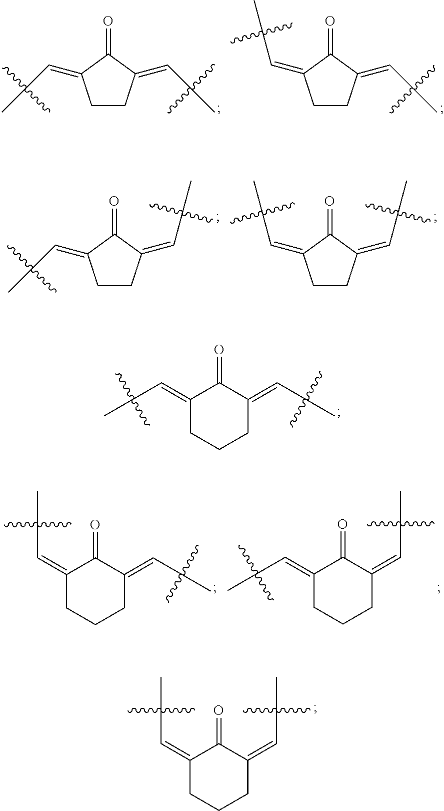

Exemplary compounds having the structure (XVII) include:

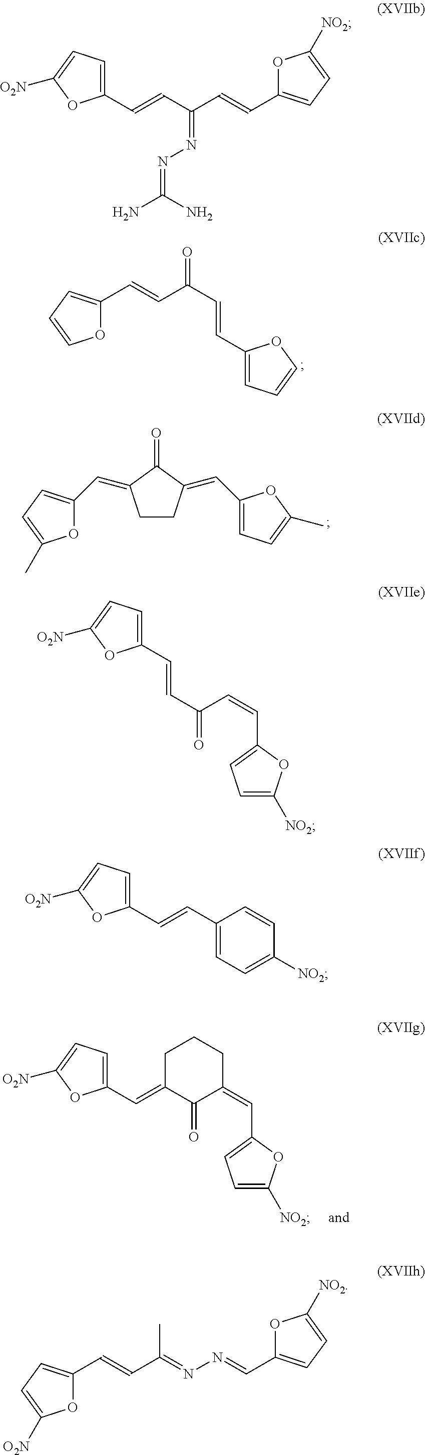

Analogues of 2-[1,5-bis(5-nitrofuran-2-yl)penta-1,4-dien-3-ylideneamino]guanidine hydrochloride that exhibit antibiotic activity include antibiotic compounds having the structure (XVIII):

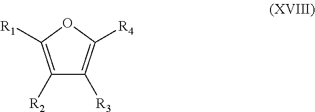

wherein:
R₁ is: —H; —NO₂; —F; —Cl; —Br; OH; —OR₈; or —CF₃
R₂ is: —H; —NO₂; —F; —Cl; —Br; OH; —OR₈; or —CF₃
R₃ is: —H; —NO₂; —F; —Cl; —Br; OH; —OR₈; or —CF₃

### R₄ is:

-(E)CH═CH—C(N—N═C(NH₂)₂)-(E)-CH═CH₂;
-(E)CH═CH—C(N—N═C(NH₂)₂)—(Z)—CH═CH₂;
—(Z)—CH═CH—C(N—N═C(NH₂)₂)-(E)-CH═CH₂;
—(Z)—CH═CH—C(N—N═C(NH₂)₂)—(X)—CH═CH₂;
-(E)-CH═CH—C(O)-(E)-CH═CH₂;
-(E)-CH═CH—C(O)—(Z)—CH═CH₂;
—(Z)—CH═CH—C(O)-(E)-CH═CH₂;
—(Z)—CH═CH—C(O)—(Z)—CH═CH₂;
-(E)CH═CH—CH═N—NH—C(S)—NH₂;
—(Z)—CH═CH—CH═N—NH—C(S)—NH₂;
-(E)-CH═CH₂—R₇; —(Z)—CH═CH₂—R₇;
-(E)CH═CH—C(Me)=N—N═CH₂;
—(Z)—CH═CH—C(Me)=N—N═CH₂;
-(E)CH═CH—C(Me)N—NH—C(NH)—NH₂;
—(Z)—CH═CH—C(Me)N—NH—C(NH)—NH₂;

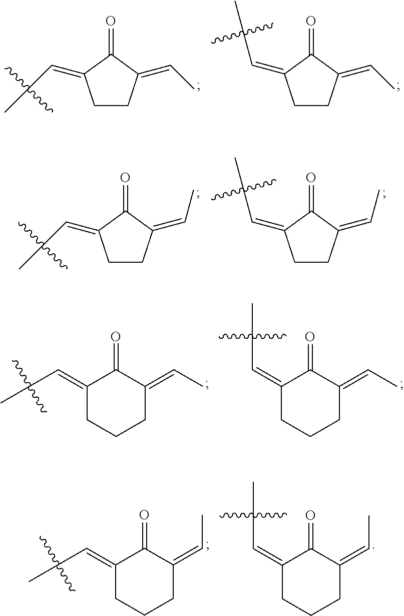

where R₇ is: —H; —NO₂; —F; —Cl; —Br; OH; —OR₈; —NH₃; —NH₂R₈; —NH(R₈)₂; —N(R₈)₃; —N(R₈)₄⁺; or —CF₃; and
R₈ is —H and C₁-C₆ alkyl.
Exemplary compounds having the structure (XVIII) include:

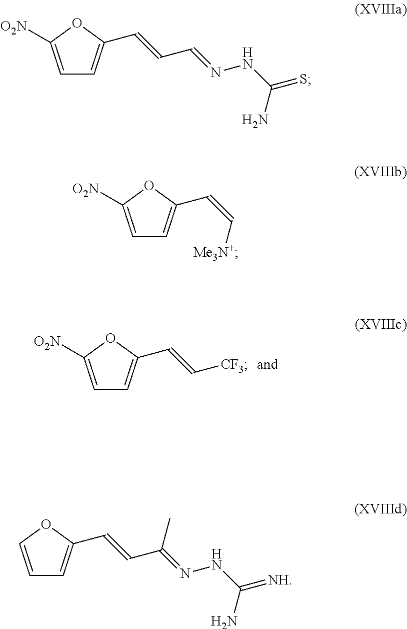

Analogues of 2-[1,5-bis(5-nitrofuran-2-yl)penta-1,4-dien-3-ylideneamino]guanidine hydrochloride that exhibit antibiotic activity include antibiotic compounds having the structure (XIX):

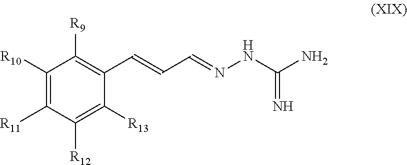

where each R₉, R₁₀, R₁₁, R₁₂, and R₁₃ are independently: —H; —NO₂; —F; —Cl; —Br; OH; —OR₈; or —CF₃;

and wherein at least one of R₉, R₁₀, R₁₁, R₁₂, and R₁₃ is not hydrogen

An exemplary compound having the structure (XIX) includes compound (XIXa):

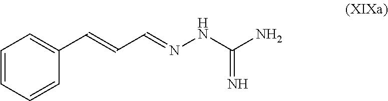 [SMILES: N=C(N)N/N=C/C=C/c1ccccc1]

Analogues of 2-[1,5-bis(5-nitrofuran-2-yl)penta-1,4-dien-3-ylideneamino]guanidine hydrochloride that exhibit antibiotic activity include antibiotic compounds having the structure (XX):

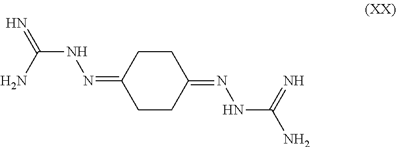 [SMILES: N=C(N)NN=C1CCC(=NNC(=N)N)CC1]

Analogues of 2-[1,5-bis(5-nitrofuran-2-yl)penta-1,4-dien-3-ylideneamino]guanidine hydrochloride that exhibit antibiotic activity include antibiotic compounds having the structure (XXI):

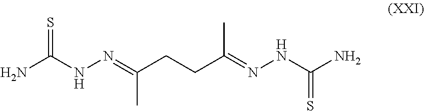 [SMILES: C/C(CC/C(C)=N/NC(N)=S)=N\NC(N)=S]

Class 6 Antibiotic Compounds—(4,6-dimethylpyrimidin-2-yl)[({4-[(4-chlorophenyl)methoxy]phenyl}amino)iminomethyl]amine Derivatives

(4,6-dimethylpyrimidin-2-yl)[({4-[(4-chlorophenyl)methoxy]phenyl}amino)iminomethyl]amine was tested in the above-described assays and found to be a ribosomal inhibitor.

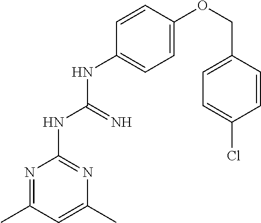 [SMILES: Cc1cc(C)nc(NC(=N)Nc2ccc(OCc3ccc(Cl)cc3)cc2)n1]

### (4,6-dimethylpyrimidin-2-yl)[({4-[(4-chlorophenyl)methoxy]phenyl}amino)iminomethyl] amine

Class 6 antibiotic compounds include (4,6-dimethylpyrimidin-2-yl)[({4-[(4-chlorophenyl)methoxy]phenyl}amino)iminomethyl]amine and derivatives and analogues of this compound.

In one embodiment, antibiotic (4,6-dimethylpyrimidin-2-yl)[({4-[(4-chlorophenyl)methoxy]phenyl}amino)iminomethyl]amine derivatives have the structure (XXII):

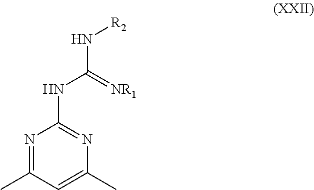

### Wherein:

R₁ is —H; —Ar; —SO₂Ar;
R₂ is: —OH; —Ar; —C(O)—CHR₃—Ar;

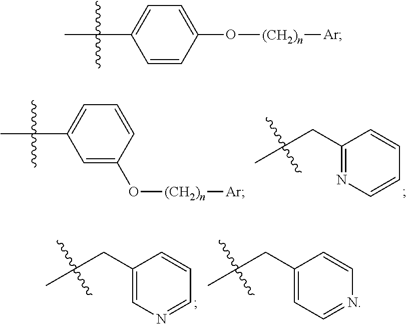

where n is 0 or 1;

### Where —Ar is:

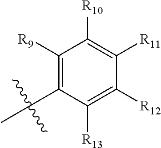

Where each of R₉, R₁₀, R₁₁, R₁₂, and R₁₃ are independently: —H; —NO₂; —F; —Cl; —Br; OH; —OR₈;—OBn; or —CF₃; where Bn is benzyl; and R₈ is C₁-C₆ alkyl.
Exemplary compounds having the structure (XXII) include:

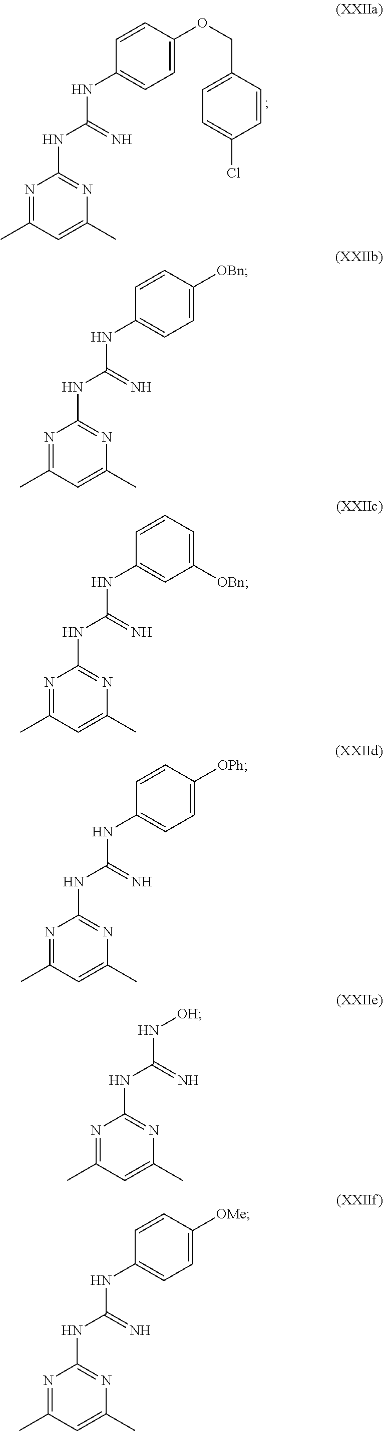

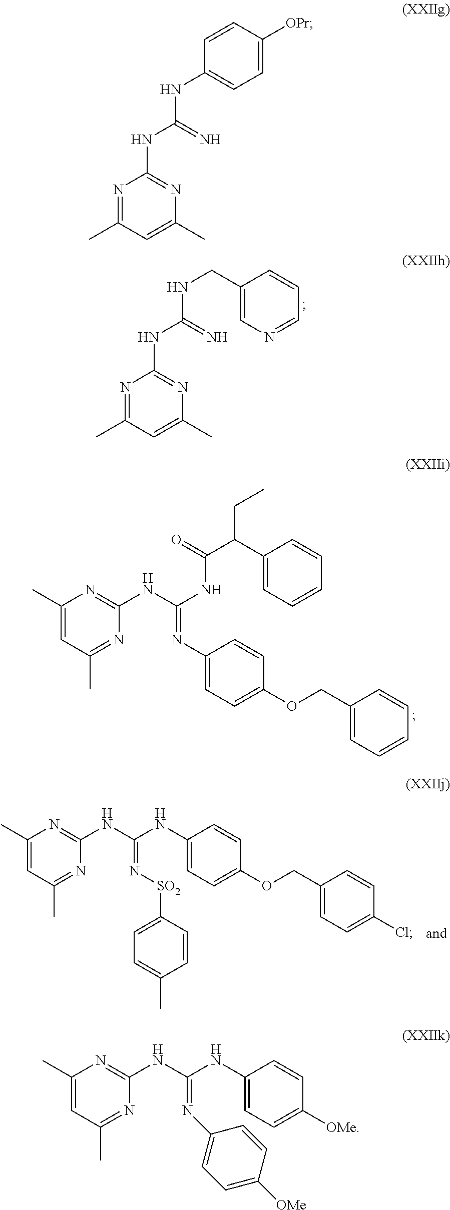

Analogues of (4,6-dimethylpyrimidin-2-yl)[({4-[(4-chlorophenyl)methoxy]phenyl}amino)iminomethyl]amine that exhibit antibiotic activity include antibiotic compounds having the structure (XXIII):

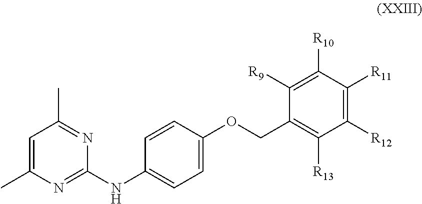

Where each of R₉, R₁₀, R₁₁, R₁₂, and Rn are independently: —H; —NO₂; —F; —Cl; —Br; OH; —OR₈;—OBn; or —CF₃; where Bn is benzyl; R₈ is C₁-C₆ alkyl; at least one of R₉, R₁₀, R₁₁, R₁₂, and R₁₃ is not hydrogen.
Exemplary compounds having the structure (XXIII) include:

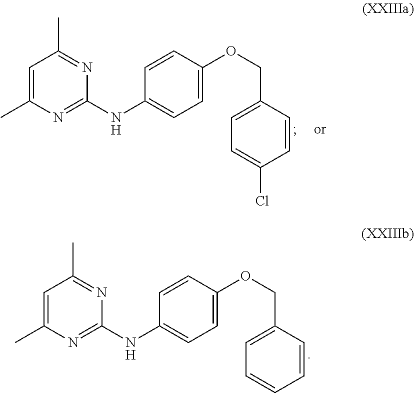 [SMILES: Cc1cc(C)nc(Nc2ccc(OCc3ccc(Cl)cc3)cc2)n1 ; Cc1cc(C)nc(Nc2ccc(OCc3ccccc3)cc2)n1]

Analogues of (4,6-dimethylpyrimidin-2-yl)[({4-[(4-chlorophenyl)methoxy]phenyl}amino)iminomethyl]amine that exhibit antibiotic activity include antibiotic compounds having the structure (XXIV):

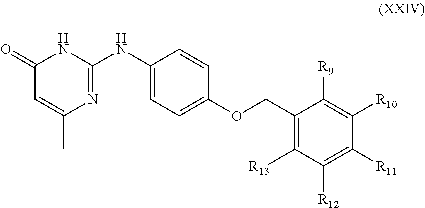

Where each of R₉, R₁₀, R₁₁, R₁₂, and R₁₃ are independently: —H; —NO₂; —F; —Cl; —Br; OH; —OR₈;—OBn; or —CF₃; where Bn is benzyl; R₈ is C₁-C₆ alkyl; and at least one of R₉, R₁₀, R₁₁, R₁₂, and R₁₃ is not hydrogen.
An exemplary compound having the structure (XXIV) is compound (XXIVa)

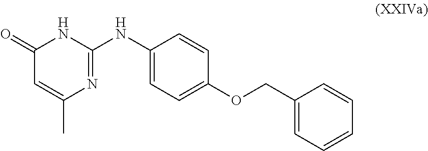 [SMILES: Cc1cc(=O)[nH]c(Nc2ccc(OCc3ccccc3)cc2)n1]

Analogues of (4,6-dimethylpyrimidin-2-yl)[({4-[(4-chlorophenyl)methoxy]phenyl}amino)iminomethyl]amine that exhibit antibiotic activity include antibiotic compounds having the structure (XXV):

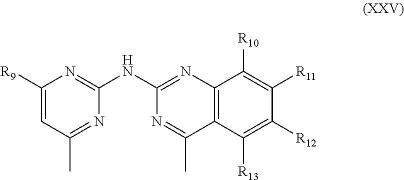

where R₉ is —H or -Me;
where each of R₁₀, R₁₁, R₁₂, and R₁₃ are independently: —H; —NO₂; —F; —Cl; —Br; OH; —OR₈;—OBn; or —CF₃; where Bn is benzyl;
R₈ is C₁-C₆ alkyl; and
at least one of R₉, R₁₀, R₁₁, R₁₂, and R₁₃ is not hydrogen.
Exemplary compounds having the structure (XXV) are:

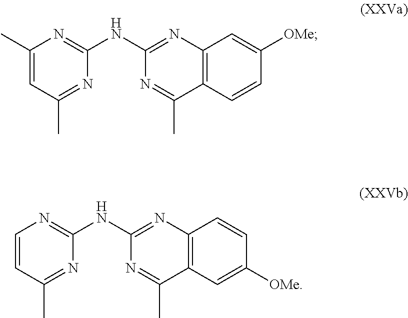 [SMILES: COc1ccc2c(C)nc(Nc3nc(C)cc(C)n3)nc2c1 ; COc1ccc2nc(Nc3nccc(C)n3)nc(C)c2c1]

Analogues of (4,6-dimethylpyrimidin-2-yl)[({4-[(4-chlorophenyl)methoxy]phenyl}amino)iminomethyl]amine that exhibit antibiotic activity include antibiotic compounds having the structure (XXVI):

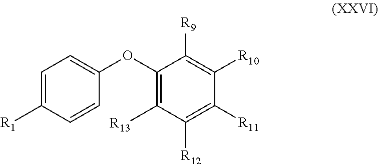

where R₁ is —NH₂; —NHR₈; —N(R₈)₂; —NH—C(O)—NH₂; —NH—C(NH)—NH₂; —NH—C(NR₈)—NH₂; —NH—C(S)—NH₂;
where each of R₉, R₁₀, R₁₁, R₁₂, and R₁₃ are independently: —H; —NO₂; —F; —Cl; —Br; OH; —OR₈;—OBn; or —CF₃; where Bn is benzyl;
R₈ is C₁-C₆ alkyl; and
at least one of R₉, R₁₀, R₁₁, R₁₂, and R₁₃ is not hydrogen.
Exemplary compounds having the structure (XXVI) are:

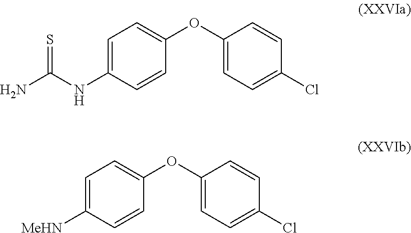 [SMILES: NC(=S)Nc1ccc(Oc2ccc(Cl)cc2)cc1 ; CNc1ccc(Oc2ccc(Cl)cc2)cc1]

Analogues of (4,6-dimethylpyrimidin-2-yl)[({4-[(4-chlorophenyl)methoxy]phenyl}amino)iminomethyl]amine that exhibit antibiotic activity include antibiotic compounds having the structure (XXVII):

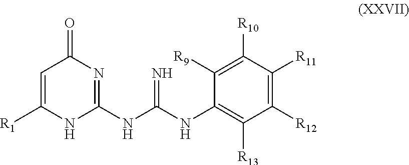

where R₁ is —H; —C₁-C₆ alkyl; or —CH₂—R₂
where R₂ is —OH; —OR₈; —CO₂H; —CO₂R₈;
where each of R₉, R₁₀, R₁₁, R₁₂, and R₁₃ are independently: —H; —NO₂; —F; —Cl; —Br; OH; —OR₈;—OBn; or —CF₃; where Bn is benzyl;
R₈ is C₁-C₆ alkyl; and
at least one of R₉, R₁₀, R₁₁, R₁₂, and R₁₃ is not hydrogen.
Exemplary compounds having the structure (XXVII) are:

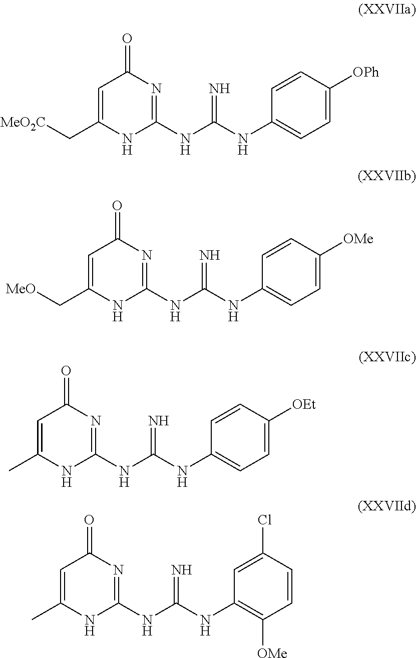 [SMILES: COC(=O)Cc1cc(=O)nc(NC(=N)Nc2ccc(Oc3ccccc3)cc2)[nH]1 ; COCc1cc(=O)nc(NC(=N)Nc2ccc(OC)cc2)[nH]1 ; CCOc1ccc(NC(=N)Nc2nc(=O)cc(C)[nH]2)cc1 ; COc1ccc(Cl)cc1NC(=N)Nc1nc(=O)cc(C)[nH]1]

### Synthesis of Antibiotic Compounds

The compounds disclosed above (compounds (I)-(XXVVII)) can be obtained commercially, synthesized using known synthetic methods, or synthesized using modifications to known synthetic methods that are within the knowledge of one of ordinary skill in the art. Exemplary methods of making compounds (I)-(XXVVII) are taught or suggested in the at least the following references, all of which are incorporated herein by reference: U.S. Pat. No. 1,921,458 to Elis et al., issued Aug. 8, 1933; U.S. Pat. No. 2,275,923 to Ross, issued Mar. 10, 1942; U.S. Pat. No. 3,153,656 to Biel, issued May 16, 1962; U.S. Pat. No. 3,433,794 to Ott et al., issued Mar. 18, 1969; U.S. Pat. No. 3,720,668 to Bruer et al., issued Mar. 13, 1973; U.S. Pat. No. 3,874,873 to Volpp, issued Apr. 1, 1975; U.S. Pat. No. 3,886,211 to Keenan, issued May 27, 1975; U.S. Pat. No. 3,925,403 to Krenzer et al., issued Dec. 9, 1975; U.S. Pat. No. 4,130,414 to Arndt et al. issued Dec. 19, 1978; U.S. Pat. No. 4,139,641 to Zeeh, issued Feb. 13, 1979; U.S. Pat. No. 4,229,581 to Heeres, issued Oct. 21, 1980; U.S. Pat. No. 5,276,058 to Satoh et al., issued Jan. 4, 1994; U.S. Pat. No. 5,675,037 to Kelly issued Oct. 7, 1997; U.S. Pat. No. 6,627,755 to Chenard, issued Sep. 30, 2003; U.S. Pat. No. 6,444,829 to Aebi et al., issued Sep. 3, 2002; U.S. Pat. No. 6,995,269 to Renhowe, issued Feb. 7, 2006; U.S. Pat. No. 7,317,034 to Sundermann, issued Jan. 8, 2008; U.S. Pat. No. 7,368,453 to Boyce et al., issued May 6, 2008; U.S. Pat. No. 7,851,654 to Park et al., issued Apr. 3, 2006; U.S. Pat. No. 7,893,096 to Valiante, Jr., issued Feb. 22, 2011; U.S. Patent Application Publication No. 2005/0065118 to Wang et al., published Mar. 24, 2005; U.S. Patent Application Publication No. 2012/0059012 to Fujii et al., published Mar. 8, 2012; PCT Published Application No. WO 92/06076 to Harrison et al., published Apr. 16, 1992; Chinese Patent Application No. 101289434 to Tao published Jun. 13, 2012; EP Patent No. 0 035 619 to Baronnet et al., published Sep. 16, 1981; and Chantarasriwong et al. “Chemistry and Biology of the Caged Garcinia Xanthones” Chemistry (2010) 16(33): 9944-9962.

### Pharmaceutical Compositions

Any suitable route of administration may be employed for providing a patient with an effective dosage of drugs of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

The compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the drugs used in the present invention are conveniently delivered in the form of an aerosol spray presentation from pressurized packs or nebulisers. The compounds may also be delivered as powders which may be formulated and the powder composition may be inhaled with the aid of an insufflation powder inhaler device.

Suitable topical formulations for use in the present embodiments may include transdermal devices, aerosols, creams, ointments, lotions, dusting powders, and the like.

In practical use, drugs used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

In some embodiments, pulmonary administration of a pharmaceutical preparation may be desirable. Pulmonary administration may include, for example, inhalation of aerosolized or nebulized liquid or solid particles of the pharmaceutically active component dispersed in and surrounded by a gas.

Possible pharmaceutical preparations, which may be used rectally, include, for example, suppositories, which consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, aqueous solutions of the active compounds in water-soluble and/or water dispersible form, for example, water-soluble salts, esters, carbonates, phosphate esters or ethers, sulfates, glycoside ethers, together with spacers and/or linkers. Suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Aqueous injection suspensions may contain substances that increase the viscosity of the suspension including, for example, sodium carboxymethyl cellulose, sorbitol, dextran, and/or cyclodextrins. Cyclodextrins (e.g., β-cyclodextrin) may be used specifically to increase the water solubility for parenteral injection of the antibiotic compounds. Liposomal formulations, in which mixtures of the antibiotic compound with, for example, egg yolk phosphotidylcholine (E-PC), may be made for injection. Optionally, the suspension may contain stabilizers, for example, antioxidants such as BHT, and/or preservatives, such as benzyl alcohol.

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress or the development of a disease state.

By way of general guidance, the daily oral dosage of each active ingredient, when used for the indicated effects, will range between about 0.001 to 1000 mg/kg of body weight, between about 0.01 to 100 mg/kg of body weight per day, or between about 1.0 to 20 mg/kg/day. Intravenously administered doses may range from about 1 to about 10 mg/kg/minute during a constant rate infusion. Compounds of this invention may be administered in a single daily dose, or the total daily dosage may be administered in divided doses of two, three, or four or more times daily.

The pharmaceutical compositions described herein may further be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

For instance, for oral administration in the form of a tablet or capsule, the pharmacologically active component may be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams or more of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

### Testing of Antibiotic Compounds

The six compounds representing the six classes of compounds originally discovered were subjected to further in vitro and in vivo testing using the assays described above. Specifically, the following compounds were tested: Class 1—(2E)-1-(2,4-Dihydroxyphenyl)-3-phenyl-2-propen-1-one; Class 2—Methyl 2,4-dichloro-3,8-dihydroxy-1,6,9-trimethyl-11-oxo-11H-dibenzo[b,e][1,4]dioxepine-7-carboxylate (“leoidin”); Class 3—{[4-chloro-3-(trifluoromethyl)phenyl]amino}-N-(3-prop-2-ynylthio(1,2,4-thiadiazol-5-yl))carboxamide; Class 4—N-[(1E)-2-(5-nitro(2-furyl))-1-azavinyl](2-hydroxy-3,5-dinitrophenyl)carboxamide; Class 5—2-[1,5-bis(5-nitrofuran-2-yl)penta-1,4-dien-3-ylideneamino]guanidine hydrochloride; Class 6—(4,6-dimethylpyrimidin-2-yl)[{4-[(4-chlorophenyl)methoxy]phenyl}amino)iminomethyl] amine.

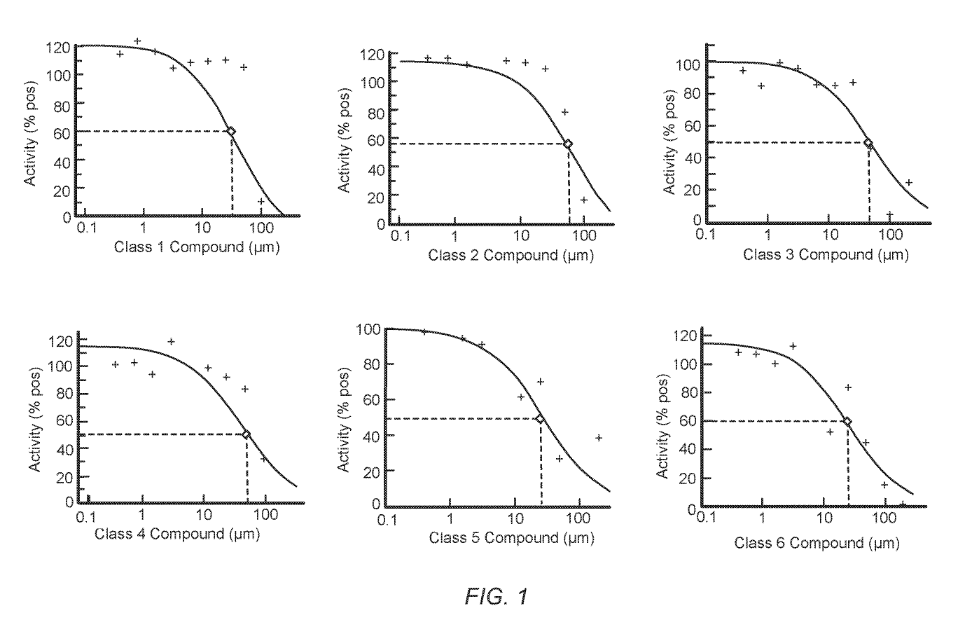

First, the concentration of the compounds causing a reduction of 50% of the activity (IC₅₀) in aminoacylation/translation (A/T) assays containing all P. aeruginosa components as described above were determined. Graphs showing the activity (% pos) vs. concentration for each representative compound of each class in an aminoacylation/translation (A/T) assay are shown in FIG. 1. All of the compounds exhibited an (IC₅₀) of less than 100 μM in aminoacylation/translation (A/T) assays.

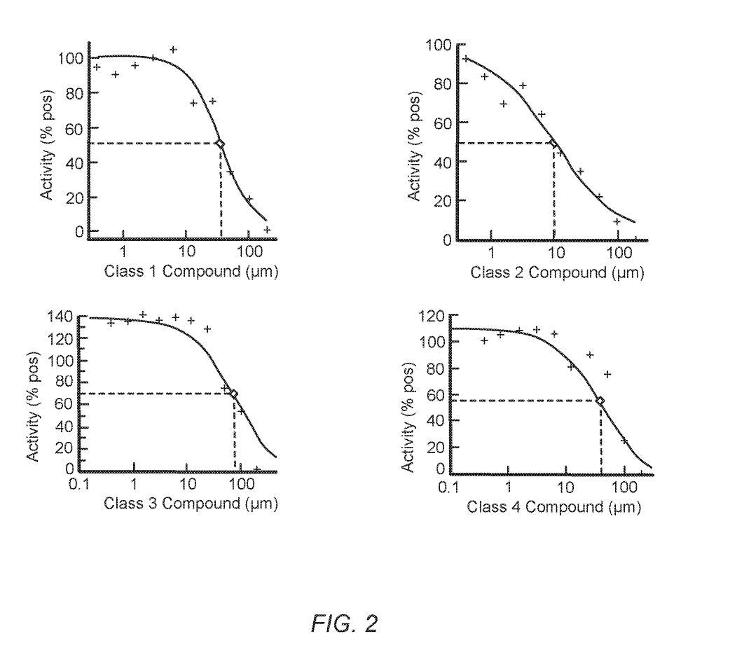

The target of four of the compounds (Classes 1-4) was determined to be PheRS, therefore we next determined the IC₅₀ for these four compounds using P. aeruginosa PheRS aminoacylation assays. Graphs showing the activity (% pos) vs. concentration for each representative compound of Classes 1-4 in a P. aeruginosa PheRS aminoacylation assay are shown in FIG. 2. All of the compounds exhibited an (IC₅₀) of less than 100 μM in P. aeruginosa PheRS aminoacylation assays.

The concentration in which 50% of the activity is inhibited (IC₅₀) in the A/T assay and in the PheRS tRNA aminoacylation assay is shown below in Table 1.

TABLE 1

| Compound | A/T Assay | PheRS Assay |
|---|---|---|
| Class 1 | 33.0¹ | 37.0 |
| Class 2 | 59.0 | 9.8 |
| Class 3 | 47.0 | 75.0 |
| Class 4 | 50.2 | 40.0 |
| Class 5 | 27.5 | N/A |
| Class 6 | 25.8 | N/A |

¹IC₅₀ values are in μM.

Next, the ability of the six compounds to affect bacterial growth in cultures was determined. We tested the compounds using broth microdilution MIC techniques performed in 96-well microtiter plates according to Clinical Laboratory Standards Institute guideline M7-A7. The results indicated that the compounds all displayed broad spectrum activity against both Gram (+) and Gram (−) organisms. The panel of bacteria included: E. coli (ATCC 25922), E. coli tolC mutant, Enterococcus faecalis (ATCC 29212), Haemophilus influenzae (ATCC 49766), Moraxella catarrhalis (ATCC 25238), Pseudomonas aeruginosa (ATCC 47085), Pseudomonas aeruginosa hypersensitive strain (ATCC 35151), Staphylococcus aureus (ATCC 29213), and Streptococcus pneumonia (ATCC 49619). These are typical pathogenic organisms used in antibacterial research. We also included mutants of both E. coli and P. aeruginosa to allow entry of the compounds into these bacterial cells which commonly have robust efflux systems for removing exogenous material. The MIC of each representative compound is shown in Table 2.

TABLE 2
Minimum inhibitory concentration (MIC) of compounds against a panel of pathogenic bacteria.

| | | E. coli | | | | | | | |
|---|---|---|---|---|---|---|---|---|---|
| | | tolC | | | | | P. aeruginosa | | |
| Compound | E. coli¹ | (efflux) | E. faecalis | H. flu | M. cat | P. aeruginosa | (hypersensitive) | S. aureus | S. pneumo |
| Class 1 | >128² | >128 | >128 | 32 | 64 | >128 | >128 | 128 | >128 |
| Class 2 | >128 | 16 | 8 | 32 | 1 | 128 | 8 | 128 | 64 |
| Class 3 | >128 | 0.5 | 128 | 2 | 128 | 32 | 4 | 1 | 8 |
| Class 4 | >128 | 2 | 128 | 32 | 32 | 32 | 32 | 32 | 128 |
| Class 5 | >128 | 2 | 128 | 16 | 64 | 128 | 4 | 16 | 32 |
| Class 6 | 64 | 16 | 16 | 32 | 64 | 64 | 16 | 32 | 32 |

¹MIC values were determined for E. coli (ATCC 25922), E. coli tolC mutant, Enterococcus faecalis (ATCC 29212), Haemophilus influenzae (ATCC 49766), Moraxella catarrhalis (ATCC 25238), Pseudomonas aeruginosa (ATCC 47085), Pseudomonas aeruginosa hypersensitive strain (ATCC 35151), Staphylococcus aureus (ATCC 29213), and Streptococcus pneumonia (ATCC 49619)
²MIC values are in μg/ml.

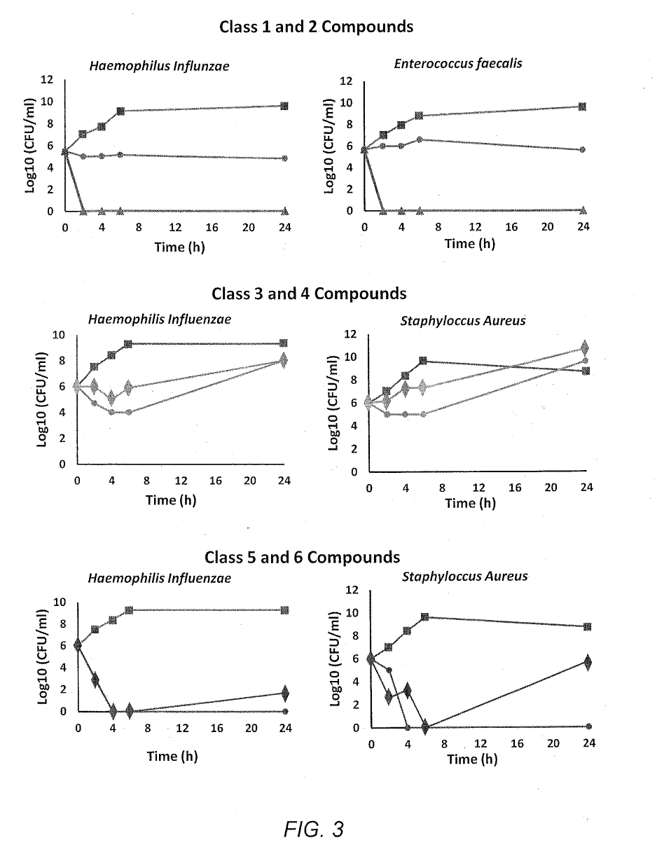

Time-kill experiments were performed according to the CLSI guideline M26-A to determine the efficacy against both Gram (+) and a Gram (−) organisms. The results are depicted in FIG. 3. The Gram (+) bacteria used was either E. faecalis or S. aureus, while the Gram (−) bacteria was H. influenzae. The (▪) lines represent bacterial growth in the absence of compound. The compounds were tested in these assays at four-times the MIC and between 0 and 24 hours. These results indicate that the: class 1 compound (▴) is bactericidal against both Gram (+) and a Gram (−) bacteria; class 2 compound () is bacteriostatic against both Gram (+) and a Gram (−) bacteria; class 3 (♦) and class 4 () compounds are bacteriostatic against both Gram (+) and Gram (−) bacteria, but at longer times losses activity; class 5 compound () is bactericidal against both Gram (+) and a Gram (−) bacteria; class 6 compound (♦) is bactericidal against both Gram (+) and a Gram (−) bacteria, but at this concentration may not have the capacity to completely kill all the bacteria present allowing some re-growth after extended time periods.

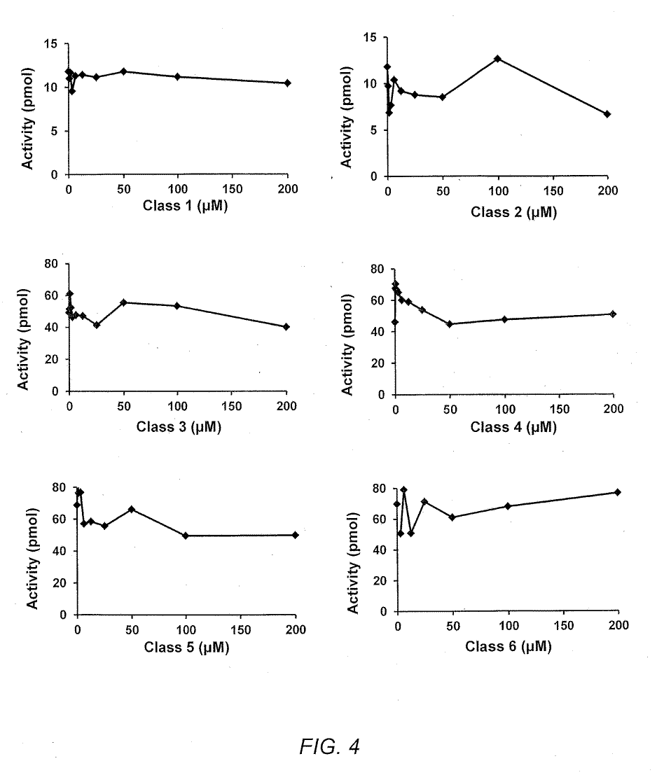

The six compounds were next tested against the protein synthesis systems from eukaryotic origins (wheat germ cell extracts). The results of these tests are depicted in FIG. 4. None of the compounds were observed to inhibit protein synthesis in these eukaryotic systems to concentrations of 200 μM.

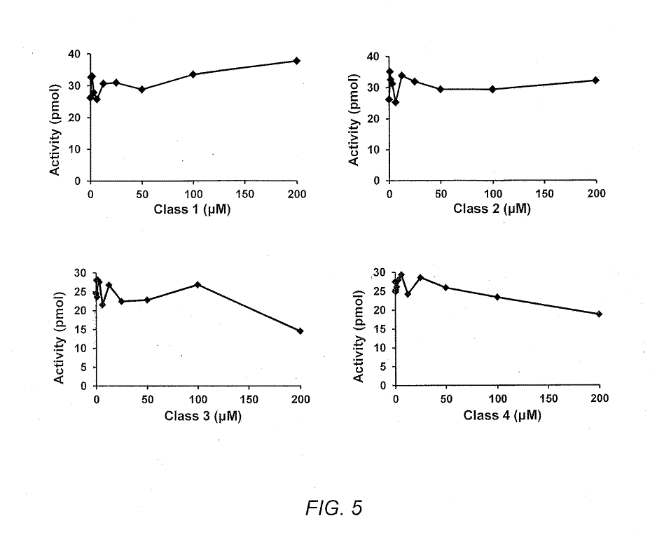

To test the inhibitory effect of the four compounds which inhibit P. aeruginosa PheRS on the activity of human mitochondrial PheRS (hmPheRS), which is a homolog of bacterial PheRS but present in eukaryotic mitochondria, aminoacylation assay containing hmPheRS were carried out. The results of these tests are depicted in FIG. 5. None of the compounds were observed to inhibit the activity of hmPheRS.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1-23. (canceled)

24. A method of treating a bacterial infection in a subject comprising administering to the subject who would benefit from such treatment a therapeutically effective amount of a pharmaceutically acceptable formulation comprising an antibiotic compound having the structure (VIII): 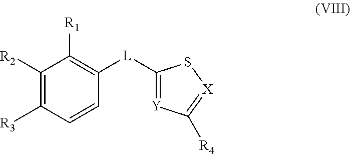
wherein:
L is: —NH—C(O)—NH—; —C(O)—NH—; —C(CH₃)—NH—C(O)—NH—; —CH₂—C(O)—NH—; —CH₂—CH(OH)—CH₂—NH—C(O)—CH₂—
X is: N; CH; CMe; C—CO₂H; C—CO₂R; C—CONH₂; C—CONHR₅; or C—CON(R₅)₂;
Y is: N; CH; CMe; C—CO₂H; or C—CO₂R; C—CONH₂; C—CONHR₅; or C—CON(R₅)₂;
R₁ is: —H; —CF₃; —R₅; —Cl; —F; —OH; or —OR₅;
R₂ is: —H; —CF₃; —R₅; —Cl; —F; —OH; or —OR₅;
R₃ is: —H; —CF₃; —R₅; —Cl; —F; —OH; or —OR₅;
R₄ is: —R₅; —S—R₅; —S—CH₂—CH═CH₂; —S—CH₂—C≡CH; phenyl; or furanyl;
R₅ is C₁-C₆ alkyl; and
wherein at least one of R₁, R₂, R₃, and R₄ is not hydrogen.

25. The method of claim 24, wherein the antibiotic compound has the structure (VIIIa): 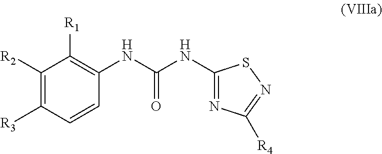
wherein:
R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅;
R₄ is: —S—R₅; and
R₅ is C₁-C₆ alkyl.

26. The method of claim 24, wherein the antibiotic compound has the structure (VIIIb): 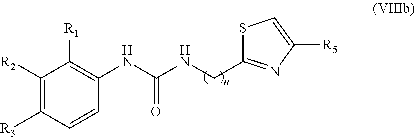
wherein:
R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅; and
R₅ is C₁-C₆ alkyl.

27. The method of claim 24, wherein the antibiotic compound has the structure (VIIIc): 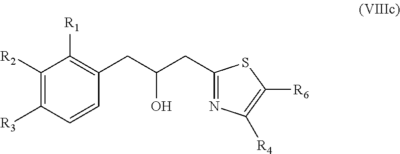
wherein:
R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅;
R₄ is: —R₅ or -Ph;
R₅ is C₁-C₆ alkyl; and
R₆ is —H; —CO₂H; —CO₂R₅.

28. The method of claim 24, wherein the antibiotic compound has the structure (VIIId): 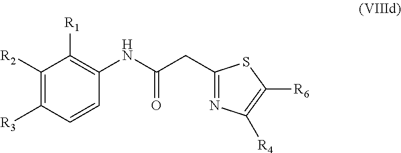
wherein:
R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅;
R₄ is: —R₅ or -Ph;
R₅ is C₁-C₆ alkyl; and
R₆ is —H; —CO₂H; —CO₂R₅.

29. The method of claim 24, wherein the antibiotic compound has the structure (VIIIe): 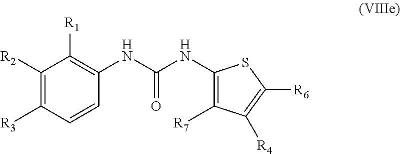
wherein:
R₁ is: —H; —CF₃;
R₂ is: —H; —CF₃; —R₅; —Cl; —F; —OH; or —OR₅;
R₃ is: —H; —CF₃; —R₅; —Cl; —F; —OH; or —OR₅;
R₄ is: —R₅; -Ph; or furanyl;
R₅ is C₁-C₆ alkyl;
R₆ is —H; —CO₂H; —CO₂R₅; —CONH₂; —CONR₅; —CON(R₅)₂; and
R₇ is —H; —CO₂H; —CO₂R₅; —CONH₂; —CONR₅; —CON(R₅)₂.

30. The method of claim 24, wherein the antibiotic compound has the structure:

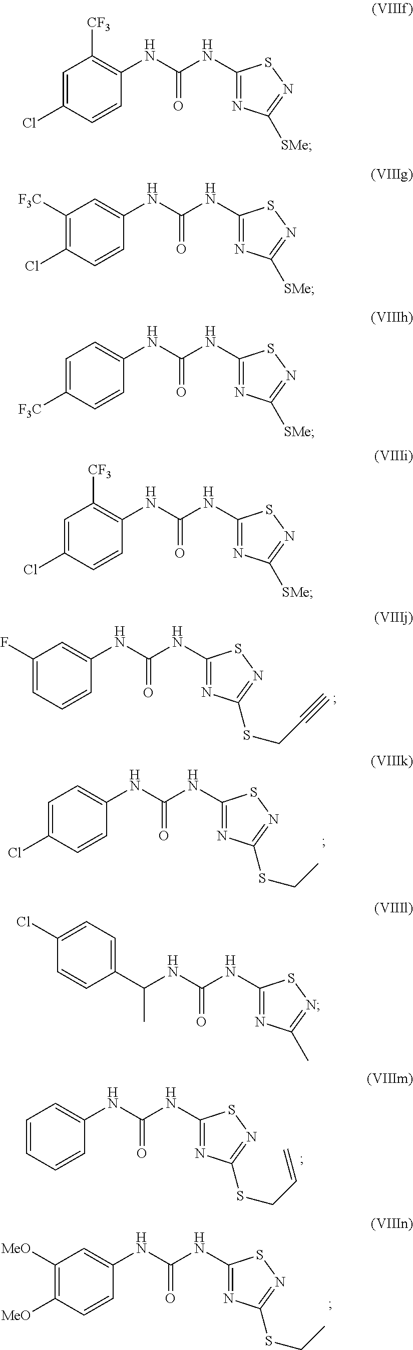

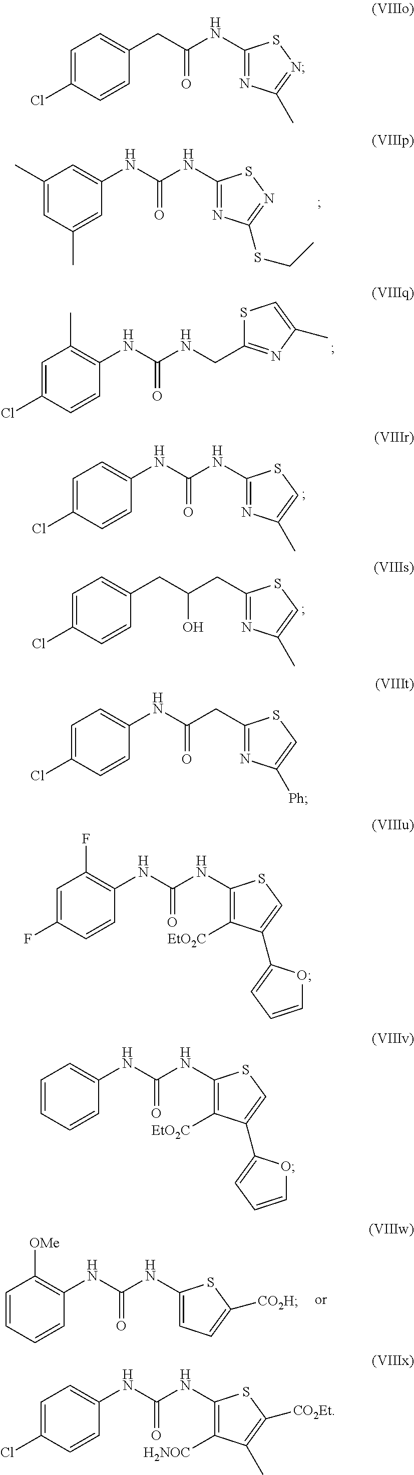

31. The method of claim 24, wherein the antibiotic compound has the structure:

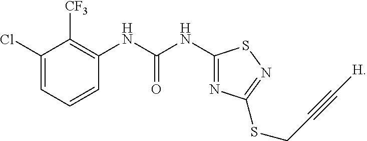

32-77. (canceled)
